(19) United States
(12) Patent Application Publication
(10) Pub. No.: US 2007/0274952 A1
(43) Pub. Date: Nov. 29, 2007

(54) Compositions and Methods for Modifying the Content of Polyunsaturated Fatty Acids in Biological Cells

(76) Inventor: Jing X. Kang, North Andover, MA (US)

Correspondence Address: EWARDS ANGELL PALMER & DODGE LLP, P.O. BOX 55874, BOSTON, MA, 02205, US

(21) Appl. No.: 10/588,406

(22) PCT Filed: Feb. 4, 2005
(86) PCT No.: PCT/US05/03917

§ 371 Date: Jun. 11, 2007

Related U.S. Application Data
(60) Provisional application No. 60/542,098, filed on Feb. 4, 2004.
(60) Provisional application No. 60/555,422, filed on Mar. 22, 2004.

(51) Int. Cl. A61K 48/00; A01K 67/00; A23L 1/31; A61P 35/00; C12N 15/00; C12N 5/00; C07H 21/04; A61P 25/00; A61K 31/7105

(52) U.S. Cl. 424/93.2; 426/641; 435/320.1; 435/325; 514/44; 536/23.5; 800/15; 800/16; 800/17; 800/19; 800/20

(57) ABSTRACT

The present invention features compositions (e.g., nucleic acids encoding fat-1, optionally and operably linked to a constitutively active or tissue-specific promoter or other regulatory sequence and pharmaceutically acceptable formulations including that nucleic acid or biologically active variants thereof) and methods that can be used to effectively modify the content of PUFAs in animal cells (i.e., cells other than those of C. elegans, for example, avian or fish cells such as myocytes, neurons (whether of the peripheral or central nervous system), adipocytes, endothelial cells, and cancer cells). The compositions and methods include a fat-1 gene that has been modified to include at least one optimized codon. The modified cells, whether in vivo or ex vivo (e.g., in tissue culture), transgenic animals containing them (fish and birds in particular), and food products obtained from those animals (e.g., meat or other edible parts of the animals (e.g., liver, kidney, or sweetbreads)) are also within the scope of the present invention.

### RELATED APPLICATIONS

This application claims the benefit of U.S. Provisional Application No. 60/542,098 filed Feb. 4, 2004, and U.S. Provisional Application No. 60/555,422, filed Mar. 22, 2004. The contents of both these applications are incorporated herein by reference in their entireties.

### GOVERNMENT SUPPORT

Some of the work presented herein was supported by a grant from the National Institutes of Health (CA79553). The United States government may, therefore, have certain rights in the invention.

### TECHNICAL FIELD

This invention relates to compositions and methods for altering the content of polyunsaturated fatty acids in animal cells.

### BACKGROUND

Polyunsaturated fatty acids (PUFAs) are fatty acids having 18 or more carbon atoms and two or more double bonds. They can be classified into two groups, n-6 or n-3, depending on the position (n) of the double bond nearest the methyl end of the fatty acid (Gill and Valivety, Trends Biotechnol. 15:401-409, 1997; Broun et al., Annu. Rev. Nutr. 19:197-216, 1999; Napier et al., Curr. Opin. Plant Biol. 2:123-127, 1999). The n-6 and n-3 PUFAs are synthesized through an alternating series of desaturations and elongations beginning with either linoleic acid (LA, 18:2n6) or α-linolenic acid (ALA, 18:3n3), respectively (Gill and Valivety, supra; Broun et al., supra; Napier et al., supra). One of the major end points of the n-6 pathway in animals is arachidonic acid (AA, 20:4n6) and major end points of the n-3 pathway are eicosapentaenoic acid (EPA, 20:5n3) and docosahexaenoic acid (DHA, 22:6n3).

An important class of enzymes involved in the synthesis of PUFAs is the class of fatty acid desaturases. These enzymes introduce double bonds into the hydrocarbon chain at positions determined by the enzyme's specificity. Although, in most cases, animals contain the enzymatic activity to convert LA (1 8:2n6) and ALA (1 8:3n3) to longer-chain PUFA (where the rate of conversion is limiting), they lack the 12- and 15desaturase activities necessary to synthesize the precursor (parent) PUFA, LA and ALA (Knutzon et al., J. Biol. Chem. 273:29360-29366, 1998). Furthermore, the n-3 and n-6 PUFA are not interconvertible in mammalian cells (Goodnight et al., Blood 58: 880-885, 1981). Thus, both LA and ALA and their elongation, desaturation products are considered essential fatty acids in the human diet. The PUFA composition of mammalian cell membranes is, to a great extent, dependent on dietary intake (Clandinin et al., Can. J. Physiol. Pharmacol. 63:546-556, 1985; McLennan et al., Am. Heart J. 116:709-717, 1988).

To the contrary, some plants and microorganisms are able to synthesize n-3 fatty acids such as ALA (18:3n-3) because they have membrane-bound 12- and 15-(n-3) desaturases that act on glycerolipid substrates in both the plastid and endoplasmic reticulum (Browse and Somerville, Annu. Rev. Plant Physiol. Plant Mol. Biol. 42: 467-506, 1991). Genetic techniques have led to the identification of the genes encoding the 12- and 15-desaturases from Arabidopsis thaliana and other higher plant species (Okuley et al., Plant Cell 6:147-158, 1994; Arondel et al., Science 258:1353-1355, 1992). Recently, a fat-1 gene encoding an n-3 fatty acid desaturase was cloned from Caenorhabditis elegans (Spychalla et al., Proc. Natl. Acad. Sci. USA 94:1142-1147, 1997; see also U.S. Pat. No. 6,194,167).

### SUMMARY

The present invention is based, in part, on the discovery that the C. elegans n-3 desaturase gene, fat-1, can be successfully introduced into other types of animal cells (e.g., the cells of mammals, birds, and fish), where it quickly and effectively elevates the cellular n-3 PUFA content and dramatically balances the ratio of n-6:n-3 PUFAs. We have also discovered modified C. elegans fat-1 nucleic acid sequences. We refer to these sequences as “optimized” when one or more of the naturally occurring codons that encode the amino acid sequence are altered but still encode the same amino acid sequence. For example, one or more of the codons represented as “GTT,” which encodes the amino acid residue valine, can be replaced with CTG, which also encodes valine; one or more of the codons represented as “CGT,” which encodes the amino acid residue arginine, can be replaced with CGC, which also encodes arginine; and so forth. The codons are modified to include codons that are preferred by the organism into which the recombinant DNA is to be inserted. Accordingly, nucleic acids that include a sequence encoding an enzymatically active protein that desaturates an omega-6 fatty acid to a corresponding omega-3 fatty acid, including nucleic acids that have been modified from an original or naturally occurring state by optimization of codon usage, and methods of using those sequences to generate transgenic animals (e.g., mammals, birds, or fish) and to treat or help prevent various diseases or conditions, are within the scope of the present invention. An optimized sequence can be incorporated in any of the vectors and cells described below, and used in any of the methods in which a wild-type sequence (e.g., a fat-1 gene) can be used. In some instances (e.g., in the production of transgenic animals), the use of a codon-optimized sequence may be preferable. Optimization is not the only way in which the nucleic acid sequences can be modified. The invention encompasses the use of sequences that encode biologically active fragments or other mutants of an omega-3 (or n-3) desaturase or an optimized omega-3 (or n-3) desaturase. Enzymatically active proteins or enzymes having omega-3 desaturase activity are proteins that, when expressed in a cell, convert n-6 PUFAs to n-3 PUFAs within the cell. These proteins or enzymes may correspond in their length and content to naturally occurring proteins (e.g., to the protein encoded by fat-1) or they may be a fragment or other mutant thereof that retains enough of the enzymatic activity to be useful in one or more of the methods described herein.

More specifically, our studies demonstrated that heterologous expression of the fat-1 gene in rat cardiac myocytes rendered those cells capable of converting various n-6 PUFAs to the corresponding n-3 PUFAs and changed the n-6:n-3 ratio from about 15:1 (an undesirable ratio) to about 1:1 (a desirable ratio). We use the term “heterologous expression” to indicate that a sequence within a given cell is not a sequence that is normally expressed in that cell; the sequence may be “heterologous” by virtue of being that of a different species. In addition, we found that an eicosanoid derived from n-6 PUFA (i.e. arachidonic acid) was significantly reduced in the transgenic cells. As described further below, levels of arachidonic acid can be assessed to determine whether a given nucleic acid encodes an enzyme having omega-3 desaturase activity; similarly, one can assess the levels of n-6 PUFA; the levels of n-3 PUFA; and/or the ratio of n-6:n-3 PUFAs. Accordingly, the present invention features compositions (e.g., nucleic acids encoding polypeptides having omega-3 desaturase activity (e.g.,fat-1)), optionally and operably linked to a constitutively active or tissue-specific promoter) and methods that can be used to effectively modify the content of PUFAs in animal cells. The cells can be mammalian, avian, or fish cells, and can be of any type that includes n-6 PUFAs. For example, the cells can be myocytes, neurons (of the peripheral or central nervous system), adipocytes, endothelial cells, hepatocytes, or cancer cells, including cancer cells of the colon, breast, liver, prostate, ovaries and cervix). While the experiments described below were conducted primarily with a C. elegans fat-1 sequence, the invention is not so limited; the compositions and methods of the invention encompass those that include (or that employ) any nucleic acid encoding a polypeptide having omega-3 desaturase activity (i.e., any polypeptide, whether naturally occurring or not, whether of C. elegans or not, that desaturates an omega-6 fatty acid to a corresponding omega-3 fatty acid). Accordingly, in specific embodiments, the invention features methods of producing n-3 fatty acids from n-6 fatty acids in an animal by producing an n-3 fatty acid desaturase (i.e., an omega-3 desaturase activity) in the animal. The production of the n-3 fatty acid desaturase can be initiated following administration, to the animal, of a nucleic acid encoding an n-3 fatty acid desaturase or a biologically active variant thereof. Sspecific examples of such nucleic acids are provided herein and similar functional sequences can be readily identified by those of ordinary skill in the art, particularly given the guidance provided herein. The animal can be of any species mentioned herein (e.g., a species within the class of mammals, birds, or fish), and may or may not have an average or “normal” ability to generate n-3 fatty acid desaturase. For example, the methods of the invention can be carried out with an animal (e.g., a human) that, prior to administration of the desaturase, has an undesirably limited ability to produce n-3 fatty acids).

The nucleic acids (e.g., a fat-1 sequence or a biologically active variant thereof) can be operably linked to a regulatory sequence. Regulatory sequences encompass not only promoters, but also enhancers or other expression control sequences, such as a polyadenylation signal, that facilitate expression of the nucleic acid in a modified cell. Such modified cells can be placed in vivo or maintained ex vivo (e.g., in tissue culture). Cells that have been removed from their natural environment are “isolated” and are within the scope of the present invention when carrying a nucleic acid sequence described herein (a nucleic acid within a heterologous cell may similarly be described as “isolated”). Non-human, transgenic animals that carry the nucleic acids or modified cells described herein are also within the scope of the present invention, as are food products obtained from those animals (e.g., meat or other edible parts of the animals (e.g., liver, kidney, skin, fat, or sweetbreads)) or generated using animal parts (e.g., broths, gravies, spreads, or any processed food made with transgenic parts (e.g., beef or chicken parts)). The food products may be prepared for human consumption or as feed for pets or livestock.

While transgenic animals are discussed further below, we note here that the animal (or any given tissue therein) may or may not be one that naturally expresses an omega-3 desaturase; the invention encompasses methods of producing n-3 fatty acids from n-6 fatty acids in animals that ordinarily lack that ability by providing them with an n-3 fatty acid desaturase via gene transfer. The methods and compositions described can also be used to enhance or supplement n-3 fatty acid desaturase activity in an animal (e.g., a mammal, bird, or fish). In one embodiment, the invention features mammalian, bird (avian), or fish (ichthyan) cells that contain a nucleic acid sequence (which may or may not be optimized, encoding an n-3 desaturase (e.g., the C. elegans n-3 desaturase) or biologically active variants (e.g., fragments or other mutants) thereof, including variants that encode the C. elegans fat-1 protein (e.g., the variant shown in FIG. 18). As noted, biologically active variants of the n-3 desaturase enzyme are variants that retain enough of the biological activity of a wild-type n-3 desaturase to be therapeutically or clinically effective (i.e., variants that are useful in treating patients, producing transgenic animals, or conducting diagnostic or other laboratory tests). For example, variants of n-3 desaturase can be mutants or fragments of that enzyme that retain at least 5-25% of the biological activity of wild-type n-3 desaturase. For example, a fragment of an n-3 desaturase enzyme is a biologically active variant of the full-length enzyme when the fragment converts n-6 fatty acids to n-3 fatty acids at least 5-25% as efficiently as does the corresponding wild-type enzyme under the same conditions (e.g., 5, 10, 20, 30, 40, 50, 75, 80, 90, 95, or 99% as efficiently as a wild-type n-3 desaturase). The conversion of n-6 fatty acids to n-3 fatty acids can also be used to help assess sequences that have been modified by codon optimization (e.g., the conversion can be used to determine whether those sequences retain biological activity or have enhanced biological activity).

Variants may also contain one or more amino acid substitutions, deletions, or additions (e.g., 1%, 5%, 10%, 20%, 25% or more of the amino acid residues in the wild-type enzyme sequence can be replaced with another amino acid residue or deleted). Said differently, a nucleic acid variants within the scope of the invention can have a sequence, or can include a sequence, that is at least 75%, 80%, 90%, 95%, or 99% identical to a wild type n-3 desaturase gene (e.g. ,fat-1 of C. elegans). Similarly, a nucleic acid variant within the scope of the invention can encode a protein having a sequence that is at least 75%, 80%, 90%, 95%, or 99% identical to a wild type n-3 desaturase (e.g., Fat-1 of C. elegans). Where the variants include substitutions, the substitutions can constitute conservative amino acid substitutions, which are well known in the art. Nucleic acid sequences that vary from wild type to the extent described here are useful in the methods of the invention so long as they remain biologically active and/or encode a protein that is biologically active.

Cells that express a fat-1 sequence (optionally, operably linked to a constitutively active or tissue-specific promoter and/or other regulatory elements) are valuable because they provide a convenient system for characterizing the functional properties of the fat-1 gene and its product. Cells in tissue culture are particularly convenient, but the invention is not so limited. Fat-1-modified cells also allow one to study any cellular mechanism mediated by n-3 fatty acids without the lengthy procedures for feeding cells or animals that are currently required. The cells can also serve as model systems that can be used, for example, to evaluate existing methods and to design new methods for effectively transferring sequences encoding an n-3 desaturase into cells in vivo.

In any of the contexts described herein (e.g., whether the compositions (e.g., nucleic acid constructs) of the invention are being used to treat patients, to generate transgenic animals, or in cell culture assays), nucleic acids encoding polypeptides having omega-3 desaturase activity (e.g., fat-1) can be co-expressed (by way of the same vector or using separate vectors of the same or different types) with another (or “second”) gene.

The second gene (the fat-1 gene being the first gene) can be, for example, another therapeutic gene (e.g., a receptor for a small molecule or chemotherapeutic agent) or a marker gene (e.g., a sequence encoding a fluorescent protein, such as green fluorescent protein (GFP) or enhanced GFP (EGFP)). Variant fat-1 nucleic acids, including optimized fat-1 nucleic acids, may encode proteins that exhibit certain advantages over their wild-type counterparts. For example, the variant sequence may be more efficacious in expressing the desaturase (in cells in culture or in vivo).

Any of the nucleic acid molecules of the invention may be “isolated” (i.e., in an environment different from the environment in which they naturally occur (see our comment below regarding the use of the qualifier “isolated” in connection with codon optimized sequences). For example, the nucleic acids may be incorporated into a plasmid or other vector, or linked to one or more heterologous sequences, including regulatory elements such as promoters and enhancers. Nucleic acid molecules are also isolated when they are contained within a heterologous cell (i.e., a cell in which they would not normally be expressed). For example, a nucleic acid containing a C. elegans fat-1 gene (or a variant thereof) in a mammalian cell (e.g., a human, bovine, or porcine cell), a bird cell (e.g., a chicken, duck, or goose cell), or a fish cell (e.g., a salmon, trout, or tuna cell) is an isolated nucleic acid. The fat-1-containing cells described here are within the scope of the present invention.

The nucleic acids of the invention can also be formulated for administration to a patient. For example, they can be suspended in sterile water or a sterile physiological buffer (e.g., phosphate-buffered saline) for oral or parenteral administration to a patient (e.g., intravenous, intramuscular, intradermal, transmucosal, transdermal, or subcutaneous injection). The formulations can also be prepared for application to the surface of a tissue or organ (e.g., as a solution, gel, or paste). In the event the patient has a tumor, the compositions can be injected into the tumor or administered to the tissue surrounding the site from which a tumor was removed.

The invention also features transgenic animals (including any animal kept as livestock or used as a food source (e.g., fish or other aquatic animals (e.g., squid, octopi, crustaceans (e.g., lobsters, crabs, snails, and shrimp), or other edible, water-living animals (e.g., eels)) that express the C. elegans n-3 desaturase gene or a biologically active variant thereof. Given the discovery that a C. elegans fat-1 gene can be efficiently expressed when delivered to an animal cell, this gene, variants thereof, and other fat-1 genescan be used to generate transgenic mice or larger transgenic animals (such as cows, pigs, sheep, goats, rabbits or any other livestock or domesticated animal; any edible bird (e.g., chicken, turkey, goose, duck, or game hen), and fish including shellfish and crustaceans) according to methods well known in the art. More specifically, the invention encompasses compositions and methods for generating transgenic fish (wherein the transgene is an omega-3 desaturase or a biologically active variant thereof (e.g., a C. elegans fat-1 gene, which may include at least one optimized codon) including cod (or any fish of the family Gadidae, order Gadiformes (e.g., haddock); halibut (the common name for either of two species of flat fish of the genus Hippoglossus); herring (the common name for several fishes of the order Clupeiformes, which also includes the anchovies); mackerel (the common name for a variety of species of imported food fishes in the family Scombridae); salmon (or any fish of the Salmonidae family, including trout); perch (or any fish of the family Percidae); shad (or any fish of the family Clupeidae); skate (or any fish of the family Rajidae); smelt (or any fish of the family Osmeridae); sole (or any fish of the family Soleidae); and tuna (or any fish of the family Scombridae).

Depending on whether the construct used contains a constitutively active promoter or a tissue-specific promoter, the omega-3 desaturase gene (e.g., a fat-1 gene) can be expressed globally or in a tissue-specific manner. The gene can be expressed in many tissue types when placed under the control of a constitutively active promoter or in a specific tissue or cell type when placed under the control of a tissue-specific promoter (e.g., a promoter that is selectively active in a specific cell type, such as a cell type within skeletal, cardiac, or smooth muscle, breast tissue, the colon, the prostate, neurons, retinal cells, pancreatic cells (e.g., islet cells), other endocrine cells, endothelial cells, skin cells, adipose cells, etc.)

The cells of the transgenic animals will contain an altered PUFA content that, as described further below, is more desirable for consumption. Thus, transgenic livestock (or any animal that is sacrificed for food (e.g., fish and game) that express the desaturase enzyme encoded by the fat-1 gene will be superior (i.e., healthier) sources of food. Food obtained from these animals or food products manufactured using these animals (e.g., processed foods and pet foods) can be provided to healthy subjects or to those suffering from one or more of the conditions described herein.

As noted, the invention features methods of treating patients (including humans and other mammals) who have, or who may develop, a condition associated with an insufficiency of n-3 PUFA or an imbalance in the ratio of n-3 :n-6 PUFAs by administering a nucleic acid encoding an n-3 desaturase or a biologically active variant thereof (e.g., a fragment, mutant, or codon optimized sequence). Alternatively, one can administer the protein encoded by the nucleic acid or biologically active variant. The treatment methods can be applied to patients who have an arrhythmia or cardiovascular disease (as evidenced, for example, by high plasma triglyceride levels or hypertension), cancer (e.g., breast cancer or colon cancer), inflammatory or autoimmune diseases (such as rheumatoid arthritis, multiple sclerosis, inflammatory bowel disease (IBD), asthma, chronic obstructive pulmonary disease, lupus, diabetes, Sjogren's syndrome transplantation, ankylosing spondylitis, polyarteritis nodosa, reiter's syndrome, or scleroderma), or a malformation (or threatened malformation, as occurs in premature infants) of the retina or brain. Suitable patients also include those having or diagnosed as having diabetes, obesity, a skin disorder, renal disease, ulcerative colitis, or Crohn's disease. Other suitable patients include those ho are at risk of rejecting a transplanted organ. Expression of fat-1 can inhibit cell death (when that death occurs, we believe, by apoptosis) in cells such as neurons, and thus the methods of the invention can also be used to treat or prevent (e.g., inhibit the likelihood of, or the duration or severity of) neurodegenerative diseases. Accordingly, the invention features methods of treating a patient who has, or who may develop, a neurodegenerative disease such as Parkinson's disease, Alzheimer's disease, Huntington's disease (HD), spinal and bulbar muscular atrophy (SBMA; also known as Kennedy's disease), dentatorubral-pallidoluysian atrophy, spinocerebellar ataxia type 1 (SCA1), SCA2, SCA6, SCA7, or Machado-Joseph disease (MJD/SCA3) (Reddy et al. Trends Neurosc. 22:248-255, 1999).

Expression of fat-1 can also increase cell death of cancer cells, and thus the methods featured in the invention can be used to treat or prevent (e.g., inhibit the likelihood of, or inhibit the severity or spread of) cancers, including breast, colon, prostate, liver, cervical, lung, brain, skin, stomach, head and neck, pancreatic, blood (e.g., leukemias and lymphomas), and ovarian cancers. In particular, these methods can be carried out with nucleic acid constructs that include an optimized C. elegans fat-1 gene and, optionally, sequences that encode a second protein (e.g., a chemotherapeutic protein). The constructs can also include regulatory sequences (e.g., constitutively active or cell type-specific promoters). Nucleic acids intended for the treatment or prevention of cancer can be formulated for administration to a patient. For example, the nucleic acids can be combined with a sterile, physiologically acceptable diluent. Where a tumor has already developed, the nucleic acids can be formulated in an injectable solution.

A balanced, or more desirably balanced, n-6:n-3 ratio is important for normal growth and development, and as noted above, the methods of the invention can be advantageously applied to patients who have no discernable disease or condition associated with PUFAs.

Abbreviations used herein include the following: AA for arachidonic acid (20:4n-6); DHA for docosahexaenoic acid (22:6n-3); EPA for eicosapentaenoic acid (20:5n-3); GFP for green fluorescent protein; Ad.GFP for adenovirus carrying GFP gene; Ad.GFP.fat-1 for adenovirus carrying both fat-1 gene and GFP gene; and PUFAs for polyunsaturated fatty acids.

Although methods and materials similar or equivalent to those described herein can be used in the practice or testing of the present invention, useful methods and materials are described below. For the purpose of any U.S. patent that may issue from the present application, all publications, patent applications, patents, and other references cited herein are incorporated by reference in their entirety.

Other features and advantages of the invention will be apparent from the following detailed description, the drawings, and the Examples.

### BRIEF DESCRIPTION OF THE DRAWINGS

FIG. 1 is a collection of four photomicrographs showing gene transfer efficiency. Rat cardiac myocytes were infected with Ad.GFP (left panels; control) or Ad.GFP.fat-1 (right panels). Forty-eight hours after infection, cardiomyocytes were visualized with bright light (upper panels) and at 510 nm of blue light (lower panels). Coexpression of GFP demonstrates visually that the transgene is being expressed in cells with a high efficiency.

FIG. 2 is an autoradiogram of a ribonuclease (RNase) protection assay of fat-1 transcript levels in cardiac myocytes infected with Ad.GFP (control) and myocytes infected with Ad.GFP.fat-1. Total RNA (10 μg) isolated from the cardiomyocytes was hybridized with anti-sense RNA probes, digested with RNase and resolved by electrophoresis through a denaturing polyacrylamide gel. The fat-1 mRNA was visualized by autoradiography. A probe targeting β-actin gene was used as control.

FIG. 3. is a pair of partial gas chromatograph traces showing fatty acid profiles of total cellular lipids extracted from control cardiomyocytes infected with Ad.GFP and cardiomyocytes infected with Ad.GFP.fat-1.

FIG. 4 is a bar graph depicting prostaglandin E₂ levels in control cardiomyocytes and cardiomyocytes expressing the fat-1 gene (as determined by enzyme immunoassay).

Values are means±SDs of three experiments and are expressed as % of control. *p<0.01.

FIG. 5 is a Table showing the polyunsaturated fatty acid composition of total cellular lipids from control cardiomyocytes and the transgenic cardiomyocytes expressing a C. elegans fat-1 cDNA.

FIG. 6 is a flowchart of an experimental protocol.

FIG. 7 is a flowchart of an experimental protocol.

FIG. 8 is a flowchart of an experimental protocol.

FIG. 9 is a pair of partial gas chromatograph traces showing fatty acid profiles of total cellular lipids extracted from control neurons and neurons infected with Ad-GFP-fat-1.

FIG. 10 is a Table comparing the PUFA composition of total cellular lipids from rat cortical neurons (control) and transgenic cells expressing a C. elegans fat-1 cDNA (fat-1).

FIG. 11 is a bar graph showing the results of an enzyme immunoassay of prostaglandin E₂ levels in control neurons and neurons expressing the fat-I gene. Ad-GFP-fat-1 infected neurons have lower levels of PGE₂ relative to control. Values are means±SD of three experiments and expressed as a percentage of control. *P<0.01.

FIG. 12 is a bar graph representing the results of an MTT assay of cell viability in control and fat-1 expressing cultures. After 24 hours of growth factor withdrawal, the cell viability of neurons expressing the fat-1 gene is 50% higher than control cells (p<0.01).

FIG. 13 is a pair of tracings showing differential responses of myocytes infected with Ad.GFP and myocytes infected with Ad.GFP.fat-1 to 7.5 mM extracellular calcium.

FIG. 14 is a line graph showing tumor volume over time (0-4 weeks after viral injection) and thus, the effect of gene transfer on tumor growth. Breast cancer cells (MDA-MB-231) were implanted subcutaneously on the back of nude mice. Three weeks later, the mice were treated with Ad.GFP-fat-1 or Ad.GFP (control; 50 μl, 10¹² VP/m) by intratumoral injection.

FIG. 15 is a table showing PUFA compositions of total cellular lipids from control MCF-7 cells and the transgenic MCF-7 cells expressing a C. elegans fat-1 cDNA.

FIG. 16 is a bar graph depicting the results of an enzyme immunoassay of prostaglandin E₂ levels in control MCF-7 cells and MCF-7 cells expressing fat-1 gene. Values are means±SE of three experiments and expressed as a percentage of control. (*P<0.05).

FIGS. 17A and 17B are representations of the nucleotide sequence of the C. elegans fat-1 cDNA and the deduced amino acid sequence of the Fat-1 polypeptide.

FIG. 18 is a representation of an optimized fat-1 nucleic acid sequence.

FIG. 19 is a pair of partial gas chromatograph traces showing the differential polyunsaturated fatty acid profiles of total lipids extracted from skeletal muscles of a wild-type mouse (WT, upper panel) and a fat-1 transgenic mouse (TG, lower panel). Both the wild-type mouse and the transgenic mouse were 8 week-old female mice, and they were fed the same diet. The levels of n-6 polyunsaturated acids (18:2n-6, 20:4n-6, 22:4n-6 and 22:5n-6) are low, whereas n-3 fatty acids (marked with *) are abundant in the transgenic muscle (lower panel) compared with the wild type muscle (upper panel). In the wild type animals, the polyunsaturated fatty acids found in the tissues are mainly (98%) the n-6 linoleic acid (LA, 18:n-6) and arachidonic acid (AA, 20:4n-6) with trace (or undetectable) amount of n-3 fatty acids. In contrast, there are large amounts of n-3 polyunsaturated fatty acids, including linolenic acid (ALA, 18:3n-3), eicosapentaenoic acid (EPA, 20:5n-3), docosapentaenoic acid (DPA, 22:5n-3) and docosahexaenoic acid (DHA, 22:6n-3), in the tissues of transgenic mice. Accordingly, the levels of the n-6 fatty acids LA and AA in the transgenic tissues are significantly reduced, indicating a conversion of n-6 to n-3 fatty acids. The resulting ratio of n-6 to n-3 fatty acids in the tissues of transgenic animals was close to 1. This n-3 rich profile of lipid with a balanced ratio of n-6 to n-3 and a more balanced AA/(EPA+DPA+DHA) can be observed in all of the organs/tissues tested, as listed in Table 1.

FIG. 20 is a pair of partial gas chromatograph traces showing the differential polyunsaturated fatty acid profiles of total lipids extracted from muscle tissue of wildtype and transgenic Zebrafish expressing the fat-1 gene, modified as described in Example 8.

FIG. 21 is a pair of partial gas chromatograph traces showing the differential polyunsaturated fatty acid profiles of total lipids extracted from tail tissue of wildtype and transgenic pigs expressing the fat-1 gene, modified as described in Example 8.

### DETAILED DESCRIPTION

The studies described below demonstrate that, inter alia, a nucleic acid molecule encoding an n-3 desaturase can be efficiently expressed in a variety of animal cell types and, as a consequence, those cells produce significant amounts of n-3 PUFA from endogenous n-6 PUFA and have a more balanced ratio of n-6 to n-3 PUFA (1:1). The studies were carried out using recombinant adenoviral expression vectors, which can mediate gene transfer in vivo or in vitro. Adenoviral vectors expressing an omega-3 desaturase (e.g. , fat-1), or biologically active variants thereof (e.g., codon optimized sequences), as well as other types of viral and non-viral expression vectors are within the scope of the invention.

More specifically, the invention features nucleic acid molecules that include a sequence encoding an enzyme that desaturates an n-6 to a corresponding n-3 fatty acid. While our studies have focused on the desaturase encoded by the C. elegans fat-1 gene, sequences encoding other desaturases can be included in the nucleic acid constructs of the invention and used in the methods of the invention. For example, the encoded desaturase can be that of a plant, a nematode other than C. elegans, cyanobacteria, or EPA-rich fungi (e.g., Saprolegnia diclina). Other fungi that can supply the desaturase sequence include Saccharomyces kluyveri and Saprolegnia diclina. Thus, the invention features nucleic acid molecules comprising a sequence encoding an n-3 desaturase operably linked to a regulatory element (e.g., a constitutively active or tissue-specific promoter). Specific promoters are known in the art and are described further below.

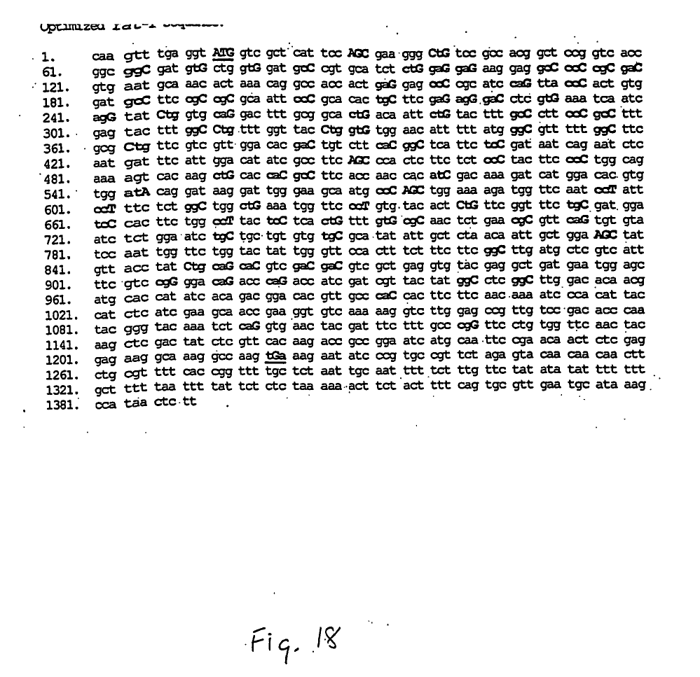

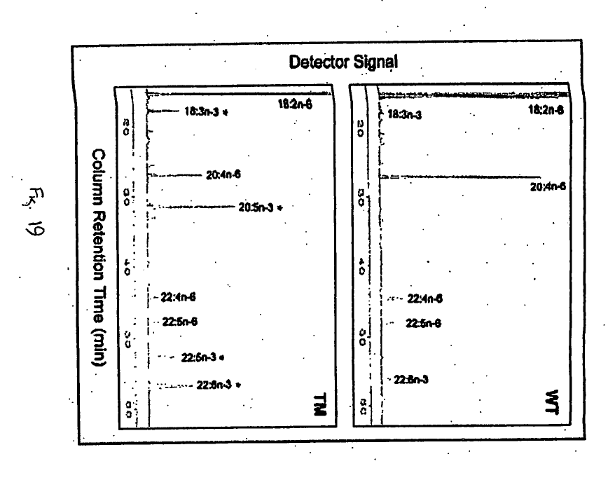

The sequence encoding the n-3 desaturase can include at least one optimized codon. The number of codons that are optimized can vary. Preferably, the number is sufficient to improve some aspect of expression (e.g., the number of copies transcribed) or to otherwise enhance the utility of the sequence. In some instances, modifying only a few codons (e.g., 1-5) can improve the sequence. In other instances, a larger number of codons (e.g., at least 5 and up to 150) can be optimized. In specific embodiments, and regardless of the initial source of the desaturase-encoding sequence, a nucleic acid molecule can include 5-10, 10-15, 15-20, 20-25, 25-30, 30-35, 35-40, 40-45, 45-50, 50-55, 55-60, 60-65, 65-70, 70-75, 75-80, 80-85, 85-90, 90-95, 95-100, 100-105, 105-110, 110-115, 115-120, 120-125, 125-130, 130-135, 135-140, 140-145, 145-150, 10-125, 25-100, 30-90, 40-80, 50-70, or about 60 optimized codons. In one embodiment, the nucleic acid molecule can include the sequence of the nucleic acid shown in FIG. 18.

Moreover, the positions of the optimized codons can vary. With respect to the C. elegans fat-1 gene, an optimized codon can be found at one or more of position 6, 9, 18, 20, 22, 24, 28-30, 33-36, 47, 49, 52, 54, 58, 60, 61, 64, 67, 69-71, 73, 77, 79, 81, 86, 89, 92, 94-95, 100, 101, 105, 106, 112, 115, 118, 124, 127, 128, 131, 146, 151, 154, 161, 163, 25 164, 169, 178, 187, 188, 195, 197, 200, 202, 206, 210, 214, 217, 221, 223, 225, 227, 228, 232, 234, 241, 245, 255, 271, 280-282, 284, 285, 301, 303, 310, 312, 327, 362, or 370. Where desaturase-encoding genes other than the C. elegans fat-1 gene are used, codons can be optimized at one or more (including all) of these same positions. In homologous genes (e.g., an n-3 desaturase gene of a plant or fungus), the positions optimized can be those corresponding to the positions listed above.

As noted above, some of the nucleic acid molecules of the invention may be referred to as “isolated”. That qualifier is not considered necessary, however, when the nucleic acid sequence is not a naturally occurring sequence. As sequences that have been optimized (particularly those in which several codons have been optimized) are highly unlikely to occur in nature, we do not see a need to refer to these sequences as “isolated”. Thus, while a nucleic acid molecule that is a naturally occurring sequence must be “isolated” (separated from some, most, or all of the components with which it is associated in its natural environment), nucleic acid molecules that are not naturally occurring (e.g., nucleic acids having an unnatural optimized sequence) need not be designated as isolated.

In addition to regulatory elements and desaturase-encoding sequences, the nucleic acid molecules can include a sequence that encodes a polypeptide that confers a benefit upon a subject to whom it is administered (e.g., a therapeutic polypeptide) or that improves the utility of the molecule in an assay (e.g., a second sequence can encode a marker protein). Examples of fluorescent (e.g., GFP and EGFP) are provided herein, as are examples of non-fluorescent marker (e.g., β-galactosidase). Other marker or “reporter” proteins are known and routinely used in the art and can also be incorporated in the nucleic acid constructs described herein.

The nucleic acid molecules can be, or can be a part of, a vector (e.g., an expression vector). We noted our use of adenoviral vectors above (see also, the Examples). Other viral vectors that can be employed as expression constructs in the present invention include vectors derived from viruses such as vaccinia virus (e.g., a pox virus or a modified vaccinia virus ankara (MVA)), an adeno-associated virus (AAV), or a herpes virus. These viruses offer several attractive features for use in connection with animal cells, including human cells. For example, herpes simplex viruses (e.g., HSV-1) can be selected to deliver a desaturase (e.g., fat-1 or a homologue thereof (or biologically active variants, including codon-optimized variants)) to neuronal cells. Such vectors are useful, for example, in treating or preventing neurodegenerative conditions.

Retroviruses, liposomes, and plasmid vectors are also well known in the art and can also be used to deliver an n-3 desaturase-encoding sequence to a cell (e.g., the expression vector pU 78 can be used when one wishes to fuse a desaturase-encoding (e.g., fat-1) sequence to the lacZ gene; lacZ encodes the detectable marker β-galactosidase (see, e.g., Ruther et al., EMBO J. 2:1791, 1983)).

As noted, a desaturase-encoding sequence (e.g. a fat-1 sequence) or a biologically active variant thereof (including a codon optimized sequence) can also be fused to other types of heterologous sequences, such as a sequence that encodes another therapeutic gene or a sequence that, when expressed, improves the quantity or quality (e.g., solubility or circulating half-life) of the fusion protein. For example, pGEX vectors can be used to express the proteins of the invention fused to glutathione S-transferase (GST). In general, such fusion proteins are soluble and can be readily purified from lysed cells by adsorption to glutathione-agarose beads followed by elution in the presence of free glutathione. The pGEX vectors (Pharmacia Biotech Inc; Smith and Johnson, Gene 67:31-40, 1988) are designed to include thrombin or factor Xa protease cleavage sites so that the cloned target gene product can be released from the GST moiety. Other fusion partners include albumin and a region (e.g., the Fc region, with or without the hinge region) of an immunoglobulin molecule (e.g., IgG, IgA, IgM, or IgE). Other useful vectors include pMAL (New England Biolabs, Beverly, Mass.) and pRIT5 (Pharmacia, Piscataway, N.J.), which fuse maltose E binding protein and protein A, respectively, to an n-3 desaturase.

Transgene expression can be sufficiently prolonged from episomal systems, so that readministration of any given expression vector, with its transgene, is not necessary. Alternatively, the vector can be designed to promote integration into the host genome, preferably in a site-specific location, which would help ensure that the transgene is not lost during the cell's lifetime. Whatever the means of delivery, transcriptional control, exerted by the host cell, would promote tissue specificity and regulate transgene expression. Accordingly, the nucleic acid molecules of the invention can include sequences that promote integration of a desaturase-encoding sequence into a host's genome.

The expression vector will be selected or designed depending on, for example, the type of host cell to be transformed and the level of protein expression desired. For example, when the host cells are mammalian cells, the expression vector can include viral regulatory elements, such as promoters derived from polyoma, Adenovirus 2, cytomegalovirus and Simian Virus 40. These regulatory elements can be used with non-mammalian (e.g., avian or fish) cells as well. The nucleic acid inserted (i.e., the sequence to be expressed; here, an n-3 desaturase, such as that encoded by fat-1) can also be modified to encode residues that are preferentially utilized in E. coli (Wada et al., Nucleic Acids Res. 20:2111-2118, 1992). Similarly, one can preferentially modify codons, if necessary or desired, in organisms other than E. coli. Modifications such as these (e.g., incorporation of various regulatory elements and codon optimization) can be achieved by standard recombinant techniques. More generally, the expression vectors of the invention can be designed to express proteins in prokaryotic or eukaryotic cells. For example, polypeptides of the invention can be expressed in bacterial cells (e.g., E. coli), fungi, yeast, or insect cells (e.g., using baculovirus expression vectors). For example, a baculovirus such as Autographa californica nuclear polyhedrosis virus (AcNPV), which grows in Spodoptera frugiperda cells, can be used as a vector to express an n-3 desaturase. While the invention is not so limited, we expect E. coli, yeast, and insect cells will serve as host cells when a primary objective of the expression is the production and purification of an omega-3 desaturase. As noted herein, the invention also encompasses expression of an omega-3 desaturase in higher order cells, including those within a wide variety of different types of transgenic animals.

As noted above, when the host cell is obtained from, or is a cell within, a multicellular animal, the expression vectors and nucleic acids used to express the desaturase (e.g., a fat-1 nucleic acid sequence) can also contain a tissue-specific promoter. Such promoters are known in the art and include, but are not limited to liver-specific promoters (e.g., albumin; Miyatake et al., J. Virol. 71:5124-5132, 1997), muscle-specific promoters (e.g., myosin light chain 1 (Shi et al., Hum. Gene Ther. 8:403-410, 1997) or α-actin), pancreatic-specific promoter (e.g., insulin or glucagon promoters), neural-specific promoters (e.g., the tyrosine hydroxylase promoter or the neuron-specific enolase promoter), endothelial cell-specific promoters (e.g., von Willebrandt; Ozaki et al., Hum Gene Ther. 7:1483-1490, 1996), and smooth muscle-cell specific promoters (e.g., 22a). Tumor-specific promoters are also being used in developing cancer therapies, including tyrosine kinase-specific promoters for B16 melanoma (Diaz et al., J. Virol. 72:789-795, 1998), DF3/MUC1 for certain breast cancers (Wen et al., Cancer Res. 53:641-651, 1993; for breast cancer, an adipose-specific promoter region of human aromatase cytochrome p450 (p450arom) can also be used (see U.S. Pat. No. 5,446,143; Mahendroo et al., J. Biol. Chem. 268:19463-19470, 1993; and Simpson et al., Clin. Chem. 39:317-324, 1993). An α-fetoprotein promoter can be used to direct expression in hepatomas (Chen et al., Cancer Res. 55:5283-5287, 1995). Where tissue-specific expression is not required or desired, the n-3 desaturase-encoding sequence can be placed under the control of (i.e., operatively linked to) a constitutively active promoter (e.g., a β-actin promoter). Other constitutively active promoters are known and used in the art.

The vectors and other nucleic acid molecules of the invention (e.g., the fat-1 cDNA per se) can also include sequences that limit the temporal expression of the transgene. For example, the transgene can be controlled by drug inducible promoters by, for example, including a cAMP response element (CRE) enhancer in a promoter and treating the transfected or infected cell with a cAMP modulating drug (Suzuki et al., Hum. Gene Ther. 7:1883-1893, 1996). Alternatively, repressor elements can prevent transcription in the presence of the drug (Hu et al., Cancer Res. 57:3339-3343, 1997). Spatial control of expression has also been achieved by using ionising radiation (radiotherapy) in conjunction with the erg1 gene promoter. Constructs that contain such regulatory sequences are within the scope of the present invention.

Host cells that include a nucleic acid molecule described herein, including an expression vector, are also within the scope of the present invention. The cells can be prokaryotic or eukaryotic. Suitable prokaryotic cells include bacterial cells, and suitable eukaryotic cells include those of mammals, birds, and fish. Cell lines, including those established and deposited in public depositories, can also be used as host cells. Any of these cells can be used, inter alia, in the process of optimizing a nucleic acid sequence. The cell may be considered healthy or diseased (e.g., the cell can be affected by inflammation or can be one that is transformed and/or proliferating at an undesirable (e.g., undesirably high) rate).

The nucleic acid molecules described herein and the proteins they encode can be included in pharmaceutical compositions. For example, the compositions can include an expression vector described herein and a physiologically acceptable diluent (e.g., normal saline or a physiologically acceptable buffer, such as phosphate-buffered saline). While human subjects are certainly intended for therapeutic or preventative measures, the invention is not so limited. The pharmaceutical compositions can be formulated for and administered to livestock, pets, zoo or circus animals, or animals found injured or ill in the wild. Regardless of the subject, the treatment can be considered successful even if it does not completely eradicate the underlying disease or condition. Ameliorating one or more signs or symptoms of a disease or slowing the progress of a disease (e.g., a neurodegenerative disease or cancer) is also beneficial and can be achieved using the compositions described herein. The nucleic acid molecule may be present in a concentrated form or in an amount suitable for administration to a subject (e.g., a therapeutically effective amount). The amount administered would be considered therapeutically effective when, upon administration to a subject, the nucleic acid expresses an n-3 desaturase to an extent that the cellular n-3 PUFA content in the subject is elevated and/or the ratio of n-6:n-3 PUFAs is more favorably balanced.

The invention also encompasses non-human transgenic animals (e.g., a mammal, a bird, or a fish) that include a nucleic acid molecule described herein. The animals may be those that are kept, bred, caught, or hunted for food (e.g., consumption by humans or other animals (e.g., livestock or pets). As noted, the mammal can be a cow, a pig, or a sheep; the bird can be a chicken, a turkey, a duck, a goose, or a game hen; and the fish can be a salmon, trout, or tuna.

Food products or dietary supplements that include these non-human transgenic animals or a tissue or processed part thereof are also within the scope of the present invention. The products may be unprocessed (as in the case of whole animals, or whole parts of animals (e.g., joints, knuckles, or organs)) or processed from a slaughtered animal or a part thereof (e.g., the bones, fat, skin, or oils obtained therefrom). Methods of making dietary supplements (e.g., fish-oil capsules) are known in the art and can be applied to the use of any of the genetically modified animals of the present invention. The invention also encompasses methods of making food products or dietary supplements from an animal described herein (e.g., a transgenic mammal, bird or fish). These methods can be carried out in any manner, including any currently known process; it is just that the source is, or includes, a non-human transgenic animal (or a part thereof), generated as described herein.

Other methods of the invention include improving the content of n-3 fatty acids in a subject's diet by administering to the subject the food product(s) or dietary supplement(s) described above. The subject may be apparently healthy or may have been diagnosed as having cancer (e.g., breast cancer, colon cancer, prostate cancer, liver cancer, cervical cancer, lung cancer, brain cancer, skin cancer, stomach cancer, head and neck cancer, pancreatic cancer, a blood cancer, or ovarian cancer). The compositions described herein can also be used in methods of inhibiting neuronal cell death in a subject by, for example, administering to the subject a therapeutically effective amount of a nucleic acid molecule described herein. Thus, the subject may be one who has been diagnosed as having a neurodegenerative disease (e.g., Alzheimer's disease, Parkinson's disease, or Huntington's disease). Other treatable or preventable conditions include an arrhythmia, cardiovascular disease, cancer, an inflammatory disease, an autoimmune disease, a malformation or threatened malformation of the retina or brain, diabetes, obesity, a skin disorder, a renal disease, ulcerative colitis, Crohn's disease, or chronic obstructive pulmonary disease. The subject may also be one who has received or who is scheduled to receive, a transplant comprising a biological organ, tissue, or cell. The method can be carried out by administering to either the subject or the transplant, a nucleic acid molecule described herein.

As noted, the nucleic acid molecules described herein (including those in which codon usage has been optimized for the host) can be used to generate non-human transgenic animals. While the invention is not so limited, we expect the nucleic acids will be used most often to genetically modify animals that are farmed or otherwise considered a source of food. The animals can be generated using techniques known in the art. More specifically, the mammals and fish can be generated using the methods described in the Examples below.

In specific embodiments, the invention features a transgenic fish or transgenic bird that includes a nucleic acid sequence encoding an enzyme that desaturates an n-6 fatty acid to a corresponding n-3 fatty acid. The transgenic fish can be: a cod or any fish of the family Gadidae, order Gadiformes; halibut; herring or any fish of the order Clupeiformes; mackerel or any fish in the family Scombridae; salmon or any fish of the Salmonidae family, including trout; perch or any fish of the family Percidae; shad or any fish of the family Clupeidae; skate or any fish of the family Rajidae; smelt or any fish of the family Osmeridae; sole or any fish of the family Soleidae; and tuna or any fish of the family Scombridae. Other fish are described above and known to those of skill in the art.

In generating the transgenic fish or bird, one can use any of the nucleic acid sequences described herein, include a nucleic acid sequence that includes a C. elegans fat-1 gene. Any of the nucleic acid sequences can include at least one optimized codon. For example, the sequence can include at least 5 and up to 150 optimized codons. In specific embodiments, and regardless of the initial source of the desaturase-encoding sequence, the nucleic acid molecule used to generate the transgenic bird or fish can include 5-10, 10-15, 15-20, 20-25, 25-30, 30-35, 35-40, 40-45, 45-50, 50-55, 55-60-65, 65-70, 70-75, 75-80, 80-85, 85-90, 90-95, 95-100, 100-105, 105-110, 110-115, 115-120, 120-125, 125-130, 130-135, 135-140, 140-145, 145-150, 10-125, 25-100, 30-90, 40-80, 50-70, or about 60 optimized codons. In one embodiment, the transgene within the fish or bird can include a nucleic acid molecule that includes the sequence of the nucleic acid shown in FIG. 18.

As in other indications, the positions of the optimized codons can vary in the constructs used to generate non-human transgenic animals (e.g., the mammals, birds, and fish described herein). With respect to the C. elegans fat-1 gene, an optimized codon can be found at one or more of position 6, 9, 18, 20, 22, 24, 28-30, 33-36, 47, 49, 52, 54, 58, 60, 61, 64, 67, 69-71, 73, 77, 79, 81, 86, 89, 92, 94-95, 100, 101, 105, 106, 112, 115, 118, 124, 127, 128, 131, 146, 151, 154, 161, 163, 164, 169, 178, 187, 188, 195, 197, 200, 202, 206, 210, 214, 217, 221, 223, 225, 227, 228, 232, 234, 241, 245, 255, 271, 280-282, 284, 285, 301, 303, 310, 312, 327, 362, or 370. Where desaturase-encoding genes other than the C. elegans fat-1 gene are used, codons can be optimized at one or more (including all) of these same positions. When homologous genes are used (e.g., an n-3 desaturase gene of a plant or fungus), the positions optimized can be those corresponding to the positions listed above.

In the examples that follow, RNA analysis and enzymatic assays were performed to assess gene expression, and gas chromatography-mass spectrometry were used to determine fatty acid profiles (these are standard techniques that one of ordinary skill in the art could use to assess any variant of the fat-1 sequence for biological activity; or incorporate in any method of assessing a sample obtained from a patient for fat-1 expression).

Some of the studies described below were conducted using cortical neurons. Fat-1 expression not only modified the cellular n-6:n-3 fatty acid ratio and eicosanoid profile in these neurons, but also protected the cells from apoptosis, thereby increasing cellular viability. More specifically, fat-1 expression modified the fatty acid ratio and protected rat cortical neurons against growth factor withdrawal-induced apoptotis in the absence of supplementation with exogenous n-3 PUFAs. Accordingly, the nucleic acid molecules (and other compositions) described herein can be used as neuroprotectants, which can be administered to premature infants and to older patients having any neurodegenerative disease (alternatively, the molecules or other compositions can be delivered to an animal, parts of which are then consumed by the patient). The protective effect of gene transfer on neuronal apoptosis mimics the protective effects of n-3 fatty acid supplementation.

The positive results obtained with neurons are especially encouraging because n-3 PUFA deficiency leads to abnormal development of the retina and the brain, particularly in premature infants (Uauy et al., Lipids 36:885-895, 2001), and animals deficient in n-3 PUFA show deficits in memory, spatial and context-dependent learning, and loss of visual acuity (Carrie et al., Neurosci. Lett. 266:69-72, 1999; Yehuda et al., J. Neurosci. Res. 56:565-70, 1999). There are also indications that various neurological disease states in humans are associated with an n-3 deficient status (Vancassel et al., Prost. Leuk. Ess. Fatt. Acids 65:1-7, 2001; Hoffman and Birch, World Rev. Nutr. Diet 83:52-69, 1998).

The biological functions of PUFAs are described further here, as these functions bear on the types of conditions amenable to treatment with the nucleic acid molecules (and other compositions) described herein. PUFAs are important structural components of membrane phospholipids and are precursors of families of signaling molecules (eicosanoids) including prostaglandins, thromboxanes, and leukotrienes (Needleman et al., Ann. Rev. Biochem. 55:69-102, 1986; Smith and Borgeat, In Biochemistry of Lipids and Membranes, D. E. Vance & J. E. Vance, Eds., Benjamin/Cummings, Menlo Park, Calif., 00 325-360, 1986). The eicosanoids derived from PUFAs play a key role in modulating inflammation, cytokine release, the immune response, platelet aggregation, vascular reactivity, thrombosis and allergic phenomena (Dyerberg et al., Lancet 2:117-119, 1978; Cyerberg and Bang, Lancet 2:433-435, 1979; James et al., Ann. J. Clin Nutr. 7:343S-3438S, 2000; Calder, Ann. Nutr. Metab. 41:203-234, 1997). The principal fatty acid precursors of these signaling compounds are arachidonic acid (AA, 20:4n6), providing an n-6 substrate that is responsible for the major synthesis of the series 2 compounds, and eicosapentaenoic acid (EPA, 20:5n3), an n-3 substrate that is responsible for the parallel synthesis of many series 3 eicosanoids with an additional double bond. The n-6:n-3 ratio in phospholipids modulates the balance between eicosaniods of the 2 and 3 series derived from AA and EPA. The eicosanoids derived from AA (series 2) and EPA (series 3) are functionally distinct and some have important opposing physiological functions (Dyerberg et al., Lancet 2:117-119, 1978; Cyerberg and Bang, Lancet 2:433-435, 1979; James et al., Am. J. Clin Nutr. 7:343S-3438S, 2000; Calder, Ann. Nutr. Metab. 41:203-234, 1997). Series 3 eicosanoids are weak agonists or, in some cases, antagonists of series 2 eicosanoids. For example, eicosanoids of the 2 series promote inflammation and platelet aggregation, and activate the immune response, whereas series 3 eicosanoids tend to ameliorate these effects. In addition, PUFAs, in the form of free fatty acids, are involved in gene expression and intercellular cell-to-cell communication (Price et al., Curr. Opin. Lipidol 11:3-7, 2000; Sellmayer et al. Lipids 31 Suppl:S37-S40, 1996; vonSchacky, J. Lab. Clin. Med. 128:5-6, 1996). Thus, PUFA can exhibit many diverse biological effects.

The compositions and methods described herein can be used to treat a variety of specific conditions as well as to improve general health, regardless of the initial state of health (e.g., poor, average, or good). Any condition that is amenable to treatment by administration of n-3 PUFAs is amenable to treatment by way of the methods of the present invention, which comprise administration of a gene encoding an n-3 desaturase (e.g., the C. elegans fat-1 gene). Some of the conditions amenable to treatment are described herein.

n-3 PUFAs have attracted considerable interest as pharmaceutical and nutraceutical compounds (Connor, Am. J. Clin. Nutr. 70:560S-569S, 1999; Simopoulos, Am. J. Clin. Nutr. 70:562S-569S, 1999; Salem et al., Lipids 31:S1-S326, 1996). During the past 25 years, more than 4,500 studies have explored the effects of n-3 fatty acids on human metabolism and health (e.g., cardiovascular health). From epidemiology to cell culture and animal studies to randomized controlled trials, the cardioprotective effects of omega-3 fatty acids have been recognized (Leaf and Kang, World Rev. Nutr. Diet. 83:24-37, 1998; De Caterina et al., Eds., n-3 Fatty Acids and Vascular Disease, Springer-Verlag, London, 1999, pp 166; O'Keefe and Harris, Mayo Clin. Proc. 75:607-614, 2000). The predominant beneficial effects include a reduction in sudden death (Albert et al., JAMA 279:23-28, 1998; Siscovick et al., JAMA 274:1363-1367, 1995), decreased risk of arrhythmia (Kang and Leaf, Circulation 94:1774-1780, 1996), lower plasma triglyceride levels (Harris, Am. J. Clin. Nutr. 65:1645S-1654S, 1997), and a reduced blood-clotting tendency (Agren et al., Prostagland. Leukot. Essent. Fatty Acids 57:419-421, 1997; Mori et al., Arterioscler. Throm. Basc. Biol. 17:279-286, 1997). Evidence from epidemiological studies shows that another n-3 fatty acid, α-linolenic acid, reduces risk of myocardial infarction (Guallar et al., Arterioscler. Thromb. Vasc. Biol. 19:1111-1118, 1999) and fatal ischemic heart disease in women (Hu et al., Am. J. Clin. Nutr. 69:890-897, 1999). Several randomized controlled trials recently have demonstrated beneficial effects of both α-linolenic acid (de Lorgeril et al., Circulation 99:779-785, 1999) and marine omega-3 fatty acids (Singh et al., Cardiovasc. Drugs Ther. 11:485-491, 1997; Von Schacky et al., Ann. Intern. Med. 130:554-562, 1999; GISSI-Prevenzione Investigators, Lancet 354:447-455, 1999) on both coronary morbidity and mortality in patients with coronary disease. The n-3 fatty acid, EPA, exerts anticancer activity in vitro and in animal models of experimental cancer (Bougnoux, Curr. Opin. Clin. Nutr. Metab. Care 2:121-126, 1999; Cave, Breast Cancer Res. Treat. 46:239-246, 1997). Human studies show that populations whose diets are rich in EPA exhibit a remarkably low incidence of cancer (Rose and Connolly, Pharmacol. Ther. 83:217-244, 1999). Supplementation with n-3 PUFAs shows therapeutic effects on inflammatory and autoimmune diseases such as arthritis (Kremer, Am. J. Clin. Nutr. 71:349S-351S, 2000; Ariza-Ariza et al., Semin. Arthritis Rheum. 27:366-370, 1998; James et al., Am. J. Clin. Nutr. 71:343S-348S), and studies with nonhuman primates (Neuringer et al., Proc. Natl. Acad. Sci. USA 83:4021-4025, 1986) and human newborns (Uauy et al., Proc. Nutr. Soc. 59:3-15, 2000; Uauy et al., Lipids 31:S167-176, 1996) indicate that the n-3 fatty acid, DHA, is essential for the normal functional development of the retina and brain, particularly in premature infants. Furthermore, n-3 PUFA have been shown to have beneficial effects on many other clinical problems, such as hypertension (Appel et al., Arch. Intern. Med. 153:1429-1438, 1993), diabetes (Raheja et al., Ann. N.Y. Acad. Sci. 683:258-271, 1993), obesity (Clarke, Br. J. Nutr. 83:S59-66, 2000), skin disorders (Ziboh, World Rev. Nutr. Diet. 66:425-435, 1991), renal disease (De Caterina et al., Kidney Int. 44:843-850, 1993), ulcerative colitis (Stenson et al., Ann. Intern. Med. 116:609-614, 1992), Crohn's disease (Belluzzi et al., N. Engl. J. Med. 334:1557-1560, 1996), chronic obstructive pulmonary disease (Shahar et al., N. Engl. J. Med. 331:228-233, 1994), and transplanted organ rejection (Otto et al., Transplantation 50:193-198, 1990). In general, a balanced n-6:n-3 ratio of the body lipids is essential for normal growth and development and plays an important role in the prevention and treatment of many clinical problems. The diseases, disorders, and conditions described above are amenable to treatment with the nucleic acid molecules (and other compositions) described herein. For example, these diseases, disorders, and conditions can be treated or prevented by administering to a subject an expression vector (e.g., as described above) that encodes an n-3 desaturase (e.g., an expression vector that includes a fat-1 gene sequence). The disease, disorder, or condition can be treated or prevented.

According to recent studies (Simopoulos, Poultry Science 79:961-970, 2000), the ratio of n-6 to n-3 essential fatty acids in today's diet is around 10-20:1. This indicates that present Western diets are deficient in n-3 fatty acids compared with the diet on which humans evolved and their genetic patterns were established (n-6/n-3=1:1) (Leaf and Weber, Am. J. Clin Nutr. 45:1048-1053, 1987). Since the n-6 and n-3 fatty acids are metabolically and functionally distinct and have important opposing physiological functions, their balance is important for homeostasis and normal development. However, n-3 and n-6 PUFAs are not interconvertible in the human body because mammalian cells lack the enzyme n-3 desaturase. Therefore, the balance between n-6 and n-3 PUFA in biological membranes is regulated based on dietary supply. Elevating the tissue concentrations of n-3 fatty acids in human subjects or animals relies on increased consumption of n-3 PUFA-enriched foods or n-3 PUFA supplements. Given the potential therapeutic actions of n-3 PUFAs, an international scientific working group has recommended diets in which the intake of n-6 fatty acids is decreased and the intake of n-3 fatty acids is increased (Simopoulos, Food Australia 51:332-333, 1999). The American Heart Association has also recently made such a dietary recommendation (AHA Dietary Guidelines: Revision 2000, Circulation 102:2284-2299, 2000).

Although dietary supplementation with n-3 PUFA is a safe intervention, it has a number of limitations. For example, to achieve a significant increase in tissue concentrations of n-3 PUFA in vivo requires a chronic intake of high doses of n-3 PUFA for a period of at least 2-3 months. Bioavailability of fatty acids to cells from the diet involves a series of physiological processes including digestion, absorption, transport and metabolism of fat. Thus, the efficacy of dietary intervention depends on the physiological and health status of an individual. A patient in critical condition or who has a gastrointestinal disorder is unlikely to be able to ingest or absorb fatty foods or n-3 PUFA supplements. In addition, encapsulated fish oil supplements are unlikely to be suited to daily use over a person's lifetime because of their high caloric content. Moreover, ingestion of some species of fish from costal waters and lakes may carry toxic amounts of mercury or organic toxins, and effective dietary intervention requires a disciplined change in dietary habits that some people may not be able to sustain. In view of the foregoing, there is a great need for the means to quickly and effectively increase cellular n-3 PUFA content and balance the n-6:n-3 ratio without resorting to long-term intake of fish or fish oil supplements. This need is met by the methods of the present invention, which create an alternative food source (via transgenic livestock whose cells contain substantially more n-3 PUFAs than in non-transgenic animals) or which provide for administration of a gene encoding an n-3 desaturase enzyme to patients (e.g., human patients). A particular advantage of the present methods is that they not only elevate tissue concentrations of n-3 PUFAs, but also simultaneously decreases the levels of excessive endogenous n-6 PUFA.

### EXAMPLES

### Example 1

### Construction of a Recombinant Adenovirus

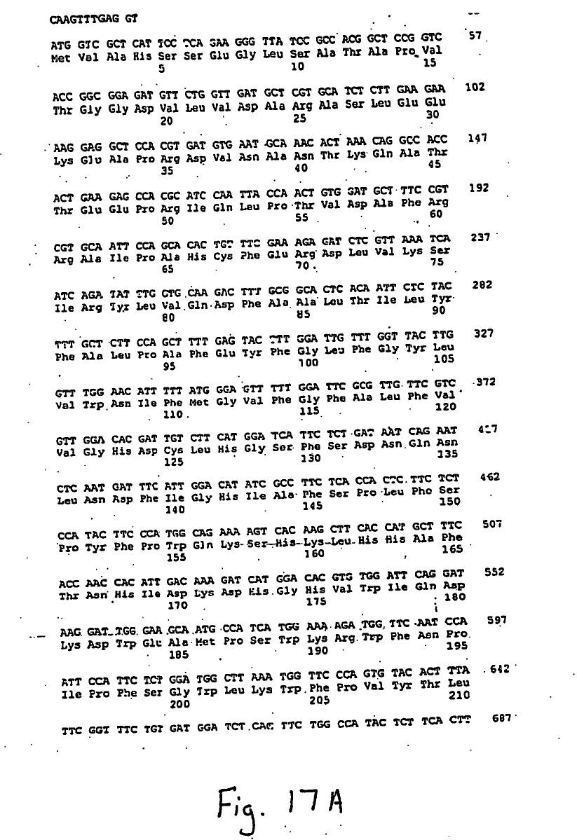

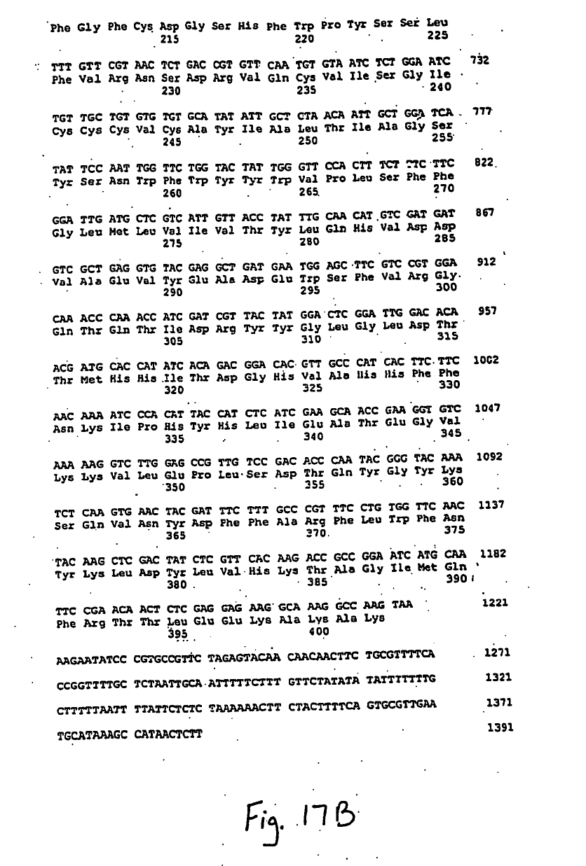

A recombinant adenovirus carrying the fat-1 gene was constructed following procedures similar to those described by He et al. (Proc. Natl. Acad. Sci. USA 95:2509-2514, 1998). The n-3 fatty acid desaturase cDNA (fat-1 gene) in pCE8 was kindly provided by Dr. J. Browse (Washington State University) (but can be synthesized or cloned using information and techniques available to those of ordinary skill in the art; see Spychalla et al., Proc. Natl. Acad. Sci. USA 94:1142-1147, 1997; U.S. Pat. No. 6,194,167; and FIG. 17A and 17B). The cDNA insert of pCE8 was excised from the plasmid with an EcoRI/KpnI double digest, inserted into a shutter vector, and then recombined with an adenoviral backbone according to the methods of He et al. (supra). Two, first-generation type 5 recombinant adenoviruses were generated: Ad.GFP, which carries the green fluorescent protein (GFP, as reporter gene) under control of the cytomegalvirus (CMV) promoter, and Ad.GFP.fat-1, which carries both the fat-1 and GFP genes, each under the control of separate CMV promoters. The recombinant viruses were prepared as high titer stocks through propagation in 293 cells, as described previously (Hajjar et al. Circulation 95:423-429, 1997). The constructs were confirmed by enzymatic digestion and by DNA sequence analysis. See also Hajjar et al., Circulation 95:4230429, 1997 and Hajjar et al., Circ. Res. 81:145-153, 1997.

Wild-type adenovirus contamination can be assessed and shown to be excluded by the absence of both PCR-detectable E1 sequences and cytopathic effects on the nonpermissive A549 cell line. Alternative adenoviral vectors with other promoters or adeno-associated viral (AAV) vectors can be constructed if necessary or desired.

### Example 2

### Culture and Infection of Cardiac Myocytes with Adenovirus.

Cardiac myocytes were isolated from one-day-old rats using the National Cardiomyocyte Isolation System (Worthington Biochemical Corp., Freehold, N.J.). The isolated cells were placed in 6-well plates and cultured in F-10 medium containing 5% fetal bovine serum and 10% horse serum at 37° C. in a tissue culture incubator with 5% CO₂ and 98% relative humidity. Cells were used for experiments after 2-3 days of culture. Viral infection was carried out by adding viral particles at different concentrations (5×10⁹-10¹⁰ pfu) to culture medium containing 2% fetal bovine serum (FBS). After a 24 hour incubation, the infection medium was replaced with normal (15% serum), culture medium supplemented with 10 μM of 18:2n-6 and 20:4n-6. About 48 hours after infection, the cells can be used (e.g., one can then analyze gene expression, fatty acid composition, viability, or growth (e.g., proliferation or rate of division)).

### Example 3

### Detecting Fat-1 Expression with Fluorescence Microscopy and RNA Analysis

Gene expression can be assessed by many methods known in the art of molecular biology. Here, expression of fat-1 in cardiac myocytes, infected as described above, was assessed by visual examination of infected cells and a ribonuclease (RNase) protection assay.

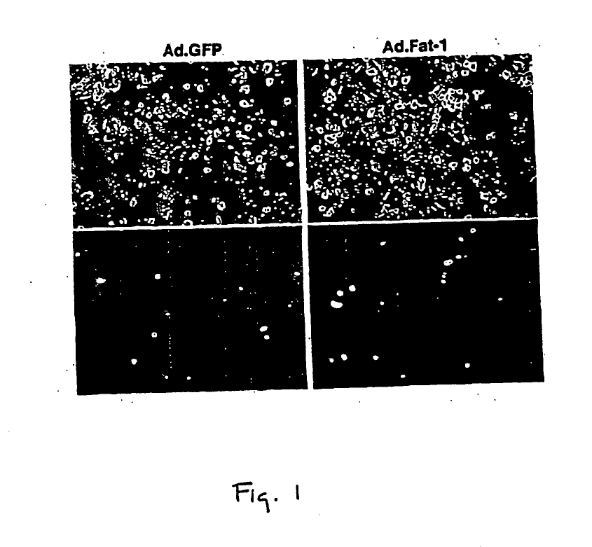

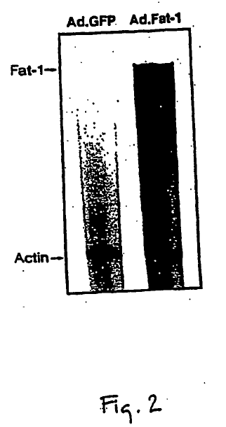

More specifically, the coexpression of GFP allowed us to identify the cells that were infected and expressed the transgene. About 48 hours after infection, almost all of the cells (>90%) exhibited bright fluorescence, indicating a high efficiency of gene transfer and a high expression level of the transgene (see FIG. 1). Expression of fat-1 transcripts was also determined by RNase protection assay using a RPA III™ kit (Ambion). Briefly, total RNA was extracted from cultured cells using an RNA isolation kit (Qiagen) according to the manufacturer's protocol. The plasmid containing the fat-1 gene, pCE8, was linearized and used as a transcription template. Anti-sense RNA probes were transcribed in vitro using ³³P-UTP, hybridized with the total RNA extracted from the myocytes, and digested with RNase to remove non-hybridized RNA and probe. The protected RNA:RNA was resolved by electrophoresis through a denaturing gel and subjected to autoradiography. A probe targeting the β-actin gene was used as a control. Fat-1 mRNA was not detected in cells infected with AD.GFP (also used as a control), but was abundant in cells infected with Ad.GFP.fat-1 (FIG. 2). This result indicates that adenovirus-mediated gene transfer confers very high expression of fat-1 gene in rat cardiac myocytes that normally lack the gene.

### Example 4

### Lipid Analysis; the Effect of n-3 Desaturase on Fatty Acid Composition

By lipid analysis, one can determine whether the expression of a fat-1 gene in cardiac myocytes (or any other cell type) converts n-6 fatty acids to n-3 fatty acids and, thereby, changes the fatty acid composition of the cell. Following infection with the adenoviruses described above, cells were incubated in medium supplemented with n-6 fatty acids (10 μM: 18:2n-6 and 10 μM 20:4n-6) for 2-3 days. After the incubation, the fatty acid composition of total cellular lipids was analyzed as described previously (Kang et al., Biochem. Bioplys. Acta. 1128:267-274, 1992; Weylandt et al., Lipids 31:977-982, 1996).

Lipid was extracted with chloroform/methanol (2:1, v/v) containing 0.005% butylated hydroxytoluene (as antioxidant). Fatty acid methyl esters were prepared using 14% BF3/methanol reagent. Fatty acid methyl esters are quantified by GC/MS using a HP5890 Series II gas chromatograph equipped with a Supelcowax SP-10 capillary column attached to a HP-5971 mass spectrometer. The injector and detector are maintained at 260° C. and 280° C., respectively. The oven program is initially maintained at 150° C. for 2 minutes, then ramped to 200° C. at 10° C./min and held for 4 minutes, ramped again at 5° C./min to 240° C., held for 3 minutes, and finally ramped to 270° C. at 10° C./min and maintained for 5 minutes. Carrier gas flow rate is maintained at a constant 0.8 mL/min throughout. Total ion monitoring is performed, encompassing mass ranges from 50-550 amus. Fatty acid mass is determined by comparing areas of various analyzed fatty acids to that of a fixed concentration of internal standard.

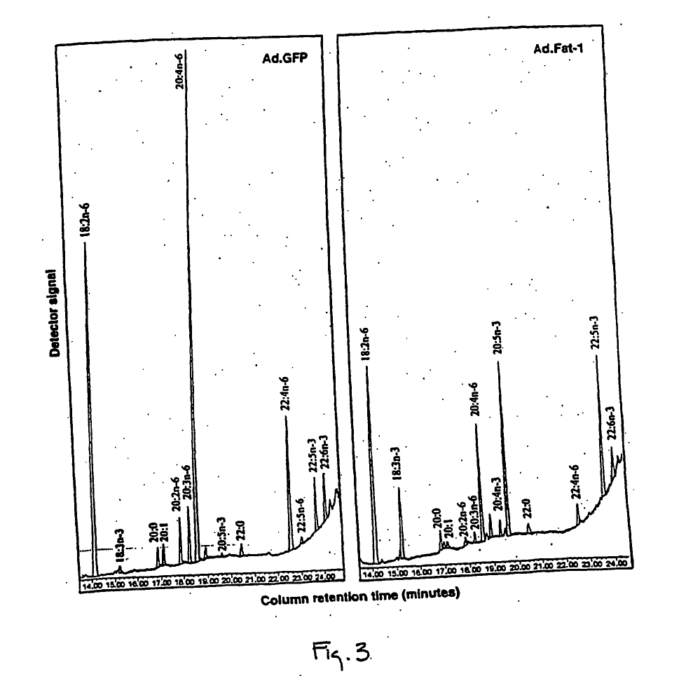

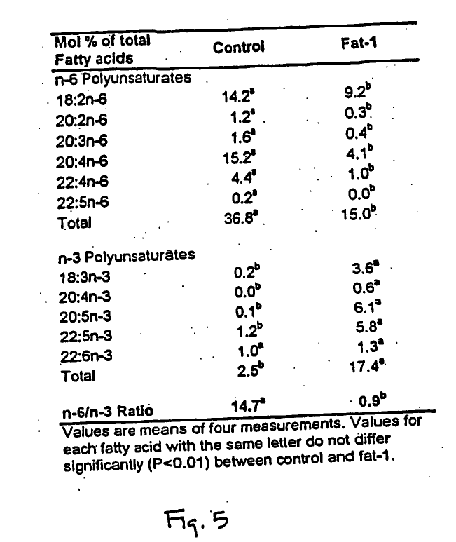

The fatty acid profiles were remarkably different between the control cells infected with Ad.GFP and the cells infected with Ad.GFP.fat-1 (FIG. 3). Moreover, cells infected with Ad.GFP showed no change in their fatty acid profiles when compared with non-infected cells. In the cells expressing the fat-1 gene (n-3 desaturase), almost all kinds of n-6 fatty acids were largely converted to the corresponding n-3 fatty acids, namely, 18:2n-6 to 18:3n-3, 20:2n-6 to 20:3n-3, 20:3n-6 to 20:4:n-3, 20:4n-6 to 20:5n-3, and 22:4n-6 to 22:5n-3. As a result, the fatty acid composition of the cells expressing fat-1 was significantly changed with respect to that of the control cells infected with Ad.GFP (FIG. 5). Importantly, the ratio of n-6:n-3 was reduced from 15:1 in the control cells to 1:1.2 in the cells expressing the n-3 fatty acid desaturase.

### Example 5

### Measuring Eicosanoids Following Fat-1 Expression

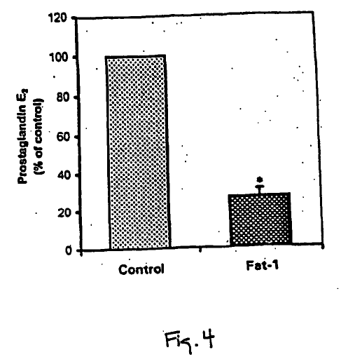

Since 20:4n-6 (AA) and 20:5n-3 (EPA) are the precursors of 2-series and 3-series of eicosanoids, respectively, differences in the contents of AA and EPA may lead to a difference in production of eicosanoids in the cells. Thus, we measured the production of eicosanoids in the infected cells following stimulation with calcium ionophore A23187 by using a EIA kit that specifically detect prostaglandin E₂ with a 16% cross-reactivity with prostaglandin E3. More specifically, Prostaglandin E₂ was measured by using enzyme immunoassay kits (Assay Designs, Inc) following the manufacturer's protocol. (The cross-reactivity with PGE3 is 16%). Cultured cells were washed and serum-free medium containing calcium ionophore A23187 (5 μM). After a 10-minute incubation, the conditioned medium was recovered and subjected to eicosanoid measurement. The amount of prostaglandin E₂ produced by the control cells was significantly higher than that produced by cells expressing the n-3 desaturase encoded by fat-1 (FIG. 4).

### Example 6

### Analysis of Animal Cells in Culture

In this example and the two that follow, we set out three different experimental models: cultured cells (other types of cultured cells are tested further below), adult rats, and transgenic mice. As shown above, the cultured cell model can be used to characterize the enzymatic properties and biochemical effects of the n-3 desaturase when expressed in mammalian cells in vitro; the adult rat model can be used to evaluate the efficacy with which a transferred fat-1 gene can elevate tissue concentrations of n-3 PUFA in vivo, and the transgenic mouse model can be used to assess the long-term and systematic effects of the transgene on lipid composition of various tissues or organs in vivo. For the first two models, the introduction of the fat-1 gene into mammalian cells/tissues will be carried out by mean of adenoviral gene transfer (mediated by recombinant adenoviruses). For the last model, gene transfer will be carried out by microinjection of the transgene into fertilized mouse eggs. Following gene transfer, the expression profile of the transferred gene can be characterized by mRNA and/or protein analysis (see, e.g., Example 3, above), and the biochemical effects, mainly the fatty acid composition of the cells or tissues, will be determined by GC-MS technology (see, e.g., Example 4, above). Eicosanoids will be measured by enzyme immunoassay (see, e.g., Example 5). Changes are identified by comparing the data obtained from fat-1-expressing cells with data obtained from control cells or tissues infected with the same (or a similar) virus, but not transfected with fat-1. The end point of these studies is the biochemical changes in cellular fatty acid composition and eicosanoid profile.

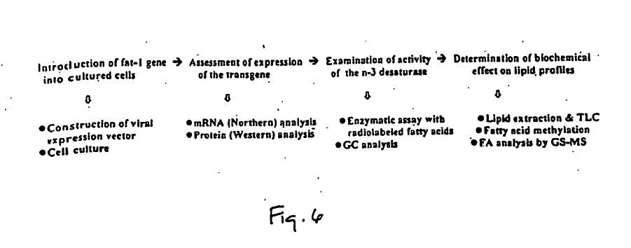

Cultures of virtually any animal cells (including human cell lines) can be infected with recombinant adenovirus (Ad.GFP.fat-1 or Ad.GFP), after which expression of the transferred gene can be assessed by RNA or protein analysis. The experimental procedures and related methods are described in the Examples above and outlined in FIG. 6. Various cell types including cardiac myocytes, neurons, hepatocytes, endothelial cells, and macrophages have been used in studies of n-3 fatty acids.

Cardiac myocytes can be isolated and cultured as described above (see Example 2), and other cell types, such as cerebellar granule neurons and hepatocytes can be prepared from 1-5 day-old rats following the method described by Schousboe et al. (In A Dissection and Tissue Culture Manual of the Nervous System, Shahar et al., Eds., Alan R. Liss, New York, N.Y., pp. 203-206, 1989). Human cell lines, including breast cancer cell lines and leukemia cell lines can be cultured in MEN medium or RPMI 1640 supplemented with 10% fetal bovine serum (FBS) in a 37° C./5% CO₂ incubator.

Viral infection can be carried out by adding viral particles at various concentrations (e.g., 2×10⁹-2×10¹⁰ pfu) to culture medium containing no FBS or 2% FBS (see also Example 2). After a 24-hour incubation, the infection medium is replaced with normal (10% FBS) culture medium. Forty-eight hours after infection, cells can be used for analysis of gene expression or fatty acid composition. Transgene expression can be assessed by fluorescence microscopy when a fluorescent tag is included in the transgene (see Example 1 and FIG. 1; similarly, the tag can be an antigenic protein detected by a fluorescent antibody) or by a standard RNA assay (e.g. a Northern blot or RNase protection assay). Since the fat-1 gene normally does not exist in control cells, it is not difficult to identify the difference in fat-1 mRNA between the control cells and cells expressing fat-1.

n-3 desaturase catalyzes the introduction of an n-3 double bond into n-6 fatty acids, leading to formation of n-3 fatty acids with one more double bond than their precursor n-6 fatty acids (e.g., 18:2n-6→18:3n-3, 20:4n-6→20:5n-3). The rate of conversion of substrates to products (the amount of products formed within a given time period) is thought to be directly proportional to the expression/activity of a desaturase. Thus, the functional activity of this enzyme can be determined, from a sample obtained from an animal (e.g., a tissue sample) or in cultured cells by measurement of the conversions (the quantity of products) using the following methods.

Fatty acid desaturation assay using radiolabeled n-6fatty acids as substrates: The assay can be performed following the protocol described by Kang et al. (Biochim. Biophys Acta. 1128:267-274, 1992). Briefly, various labeled n-6 fatty acids (e.g., [¹⁴C]18:2n-6, [¹⁴C]20:4n-6) bound to BSA are added to serum-free culture medium and incubated with cells for 4-6 hours. After that, cells and culture medium will be harvested. Lipids are extracted and methylated (see below). The labeled fatty acid methyl esters are separated according to degree of unsaturation (i.e., the number of double bond) on silica-gel TLC plates impregnated with AgNO₃. Bands containing fatty acids with different double bonds can be identified by comparison with reference standards. Quantity of the labeled fatty acids is determined by scintillation counting, and data are compared between control cells and the cells expressing the fat-1 gene.

Fatty acid analysis by gas chromatography: Conversion of fatty acids can be determined more accurately by analysis of fatty acid composition using gas chromatography-mass spectrometry (see below). Using this method, no radiolabeled fatty acid is required. Fatty acid contents of cultured cells expressing the n-3 desaturase gene, in the presence of various substrates, can be analyzed. The conversion of each fatty acid can be determined by comparison of fatty acid profiles between control cells and the cells expressing the fat-1 gene.

The fatty acid composition of total cellular lipids or phospholipids can be analyzed as described previously (Kang et al., Biochim. Biophys. Acta. 1128:267-274, 1992; Weylandt et al., Lipids 31:977-982, 1996). The procedures are as follows:

Lipid extraction (see also Example 4): Five ml of chloroform/methanol (2: 1, v/v) containing 0.005% butylated hydroxytoluene (as antioxidant) is added to washed cell pellets and vortexed vigorously for 1 minute then left at 4° C. overnight. One ml of 0.88% NaCl is added and mixed again. The chloroform phase containing lipids is collected. The remains are extracted once again with 2 ml chloroform. The chloroform is pooled and dried under nitrogen and stored in sealed tubes at −70° C.

Separation of lipids by thin-layer chromatography (TLC): TLC plates are activated at 100° C. for 60 minutes. TLC tanks are equilibrated with solvent for at least one hour prior to use. Total phospholipid and triacyglycerol are separated by running the sample on silica-gel G plates using a solvent system comprised of petroleum ether/diethyl ether/acetic acid (80:20:1 by vol.) for 30-35 minutes. Individual phospholipids are separated by TLC on silica-gel H plates using the following solvent system: chloroform/methanol/2-propanol/0.25% KCl/triethylamine (30:9:25:6:18 by vol.). Bands containing lipids are made visible with 0.01% 8-anilino-1-naphthalenesulfonic acid, and gel scrapings of each lipid fraction are collected for methylation.

Fatty acid methylation: Fatty acid methyl esters are prepared using 14% BF₃/methanol reagent. One or two ml of hexane and 1 ml of BF₃/methanol reagent are added to lipid samples in glass tubes with Teflon-lined caps. After being flushed with nitrogen, samples are heated at 100° C. for one hour, cooled to room temperature and methyl esters are extracted in the hexane phase following addition of 1 ml H₂O. Samples are allowed to stand for 20-30 minutes, the upper hexane layer is removed and concentrated under nitrogen for GC analysis.

Gas chromatography-mass spectrometry. Methylated samples are reconstituted in 100-200 μl hexane or isooctane of which 1-2 μl will be analyzed by gas chromatography. An Omega was column (30 m; Supelco, Bellefonte, Pa.) will be used in a Hewlett-Packard 5890A gas chromatograph (Hewlett-Packard, Avondage, Pa.). Carrier gas is hydrogen (2.39 ml/min), injected with a split ratio of 1:31. The temperature is initially 165° C. for 5 minutes, then is increased to 195° C. at 2.5° C./min and, from there, to 220° C. at 5° C./min. The temperature is held for 10.5 minutes and then decreased to 165° C. at 27.5° C./min. Peaks will be identified by comparison with fatty acid standards (Nu-Chek-Prep, Elysian, Minn.), and area percentage for all resolved peaks will be analyzed using a Perkin-Elmer M1 integrator (Perkin-Elmer, Norwood, Conn.). These analytical conditions separates all saturated, mono, di- and polyunsaturated fatty acids from C14 to C25 carbons in chain length. The sample size will be calculated based on external standards when added. In addition, the gas chromatography-mass spectrometry (GC-MS) will be carried out using a Hewlett-Packard mass selective detector (model 5972) operating at an ionization voltage of 70 eV with a scan range of 20-500 Da. The mass spectrum of any new peak obtained will be compared with that of standards (Nu Chek Prep, Elysian, Minn.) in the database NBS75K.L (National Bureau of Standards).

### Example 7

### Evaluation of n-3 Desaturase Gene Transfer in vivo

The experiments described here allow introduction of the fat-1 gene into animal tissues or organs (e.g., heart), where the enzyme product can quickly optimize fatty acid profiles by increasing the content of n-3 PUFAs and decreasing the content of n-6 PUFAs. The heart is selected as an experimental target for the gene transfer because it has been well studied in relation to n-3 fatty acids, and it is a vital organ.

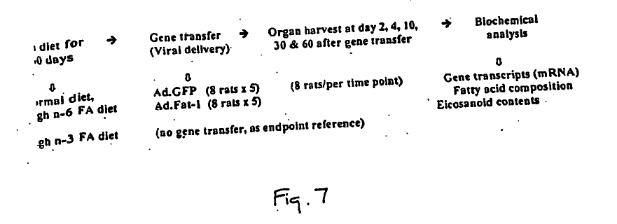

Adult rats, fed a normal diet or a diet high in n-6 PUFA for two months, will be randomized to receive either an adenovirus carrying the fat-1 gene (Ad.GFP.fat-1) or an adenovirus carrying the reporter gene GFP (Ad.GFP, as control). The adenoviruses will be delivered to the heart of a living animal using a catheter-based technique, which can produce an expression pattern that is grossly homogeneous throughout the heart (Hajjar et al., Proc. Natl. Acad. Sci. USA 95:525105256, 1998). Two days, 4 days, 10 days, 30 days and 60 days after infection (gene transfer), animals will be sacrificed, and their hearts will be harvested and used for determination of the transgene expression and analysis of fatty acid composition. Another group of rats will be fed a diet rich in n-3 fatty acids (low n-6/n-3 ratio) for two months without gene transfer and used as references. These experiments (in which animals are on different diets and samples harvested at different time points) are designed to determine whether transfer of the fat-1 gene can bring about a desired biochemical effect (n-6/n-3 ratio, eicosanoid profile) similar to or even superior to that induced by dietary intervention (i.e., n-3 FA supplementation), how quickly a significant change in fatty acid composition can be achieved, and how long the change can last. Rats injected with the reporter (GFP) gene will be used as controls (our preliminary studies showed that gene transfer of GFP has no effect on fatty acid composition). The experimental flow chart is shown in FIG. 7.

Animals and Diets: weight-matched adult Sprague-Dawley rats will be randomly assigned to three groups. Each group is fed with one of three different diets: normal (basal) diet, a high n-6 diet, or a high n-3 diet. These diets are prepared as follows.

Basal diet: a commercial rat fat-free diet (Agway Inc. C.G., Syracuse, N.Y.) to which 2% (w/w) corn oil is added; High n-6 diet: the basal diet supplemented by addition of a further 13% (w/w) corn oil or safflower oil (high in n-6 fatty acids), bringing the final diet to a total of 15% fat; High n-3 diet: the basal diet supplemented with 13% (w/w) fish oil (30% EPA, 20% DHA, 65% total n-3 PUFA) (Pronova Biocare A/S, Oslo), bringing the final diet to a total of 15% fat. This group will serve as a control group for this study.

The diets will be prepared in small batches weekly, kept at −20° C. and thawed daily in the amounts required. Vitamin E (100 mg/100 g fat) and butylated hydroxy toluene (final concentration 0.05%) will be added to prevent oxidation of long-chain polyunsaturated fatty acids (The BHT should serve to prevent autooxidation of the unsaturated fatty acids during preparation and storage). To ensure animals are receiving adequate nutrition, the rats in all groups will be weighted weekly. After 8 weeks on the diets, the animals will be subjected to gene transfer.

Adenoviral Delivery Protocol. The delivery of adenoviruses to the heart will be performed by using a cathether-based technique similar to that described by Hajjar et al. (supra). Briefly, rats will be anesthetized with intra peritoneal pentobarbital (60 mg/kg) and placed on a ventilator. The chest is entered from the left side through the third intercostals space. The pericardium is opened and a 7-0 suture placed at the apex of the left ventricle. The aorta and pulmonary artery are identified. A 22-gauge catheter containing 200 μL adenovirus (9-10×10¹⁰ pfu/ml) is advanced from the apex of the left ventricle (LV) to the aortic root. The aorta and pulmonary arteries are clamped distal to the site of the catheter, and the solution is injected. The clamp is maintained for 10 seconds while the heart pumped against a closed system (isovolumically). After 10 seconds, the clamp on the aorta and pulmonary artery is released, the chest is closed, and the animals are extubated and transferred back to their cages.

At day 2, 4, 10, 30 and 60 after gene transfer, animals will be sacrificed, their hearts infected with the viruses will be removed, perfused or rinsed with saline to removed all blood and a portion of the tissues will be promptly frozen at −80° C. for lipid analysis and eicosanoid measurement. The remaining tissues will be used for determination of the mRNA levels and/or protein levels of the n-3 desaturase.

It is possible that other organs such as brain and liver may also be infected at high levels by the adenoviruses entering the blood stream. Thus, other organs, in addition to the heart, will be also harvested for analyses of transgene expression and lipid profile.

Other methods, including assessment of transgene expression (by Northern blot, RNase protection assay, or in situ hybridization), analysis of fatty acid composition, measurement of eicosanoids, and statistical analysis will be carried out, as described above in the context of cultured cells.

### Example 8

### Transgenic Animals

The studies described here are designed to create transgenic mice that globally express the fat-1 gene and to characterize the tissue and organ lipid profiles of these animals. Transgenic mice have become a valuable model for evaluation of physiological significance of a gene in vivo. Availability of transgenic mice allows us to study the effect of a transgene in a variety of cell types at different stages of an animal's lifespan. This n-3 transgenic mouse model will provide new opportunities to elucidate the roles of n-3 PUFA and compounds derived from them in the development and cell biology.

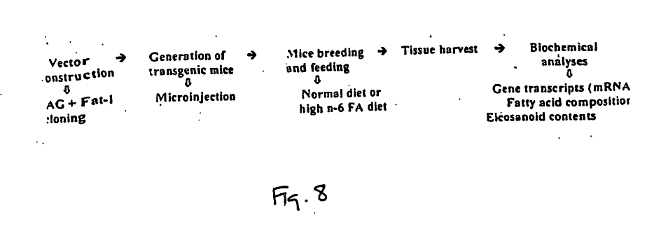

To generate transgenic animals that can globally express the fat-1 gene, one can use an expression vector that contains a fat-1 gene and the chicken beta-actin promoter with the CMV enhancer (CAG promoter), which is highly active in a wide range of cell types and therefore allows high-level and broad expression of the transgene (Niwa et al., Gene 108:193-199, 1991; Okabe et al., FEBS Lett. 407:313-319, 1997) (transgenic fish are specifically described below). The expression construct will be microinjected into the pronuclei of one-cell embryos of C57BL/6×C3H mice to produce transgenic mice. They will be bred and transgenic mouse line is established. Weanling mice are fed either a normal diet or a diet high in n-6 PUFA. Various tissues will be harvested from these animals at different ages (neonate, wean—1 month, adult—6 ms and aging—12 ms, 3-5 mice per time point will be used) for assessment of the expression levels of the transgene and determination of fatty acid composition. The levels of eicosanoids in plasma and various tissues will also be measured. A group of wild-type mice (C57BL/6) fed with the same diet (either a normal diet or a high n-6 diet) will be used as controls. The results will be compared with those from wild type animals fed the same diet. The procedure is illustrated in FIG. 8.

The transgene will be prepared by methods similar to those described by Okabe et al. (supra). Briefly, a cDNA encoding the fat-1 gene is amplified by PCR with primers, 5-agaattcggcacgagccaa gtttgaggt-3′ (SEQ ID NO: 1) and 5′-gcctgaggctttatgcattcaacgcact-3′ (SEQ ID NO:2), using pCE8-fat1 (provided by Dr. J. Browse, Washington State University) as a template. No additional amino acid sequence is added on either side of the fat-1. The PCR product will be confirmed by DNA sequencing. The EcoR1 and Bgl-II sites included in the PCR primers are used to introduce the amplified fat-1 cDNA into a pCAGGS expression vector containing the chicken beta-actin promoter and cytomegalovirus enhancer, beta-actin intron and bovine globin poly-adenylation signal (provided by Dr. J Miyazaki, Osaka University Medical School). The entire insert with the promoter and coding sequence will be excised with BamHI and Sal1 and gel-purified.

Transgenic mouse lines will be produced by injecting the purified BamHI and SalI fragment into C57BL/6×C3H fertilized eggs. The DNA-injected eggs are transplanted to pseudo-pregnant mice (B6C3F1) to produce transgenic mice. The founder transgenic mice will be identified by PCR and Southern blot analyses of tail DNA and bred with C57BL/6J mice. Offspring (either heterozygote or homozygote) will be used dependent on the expression levels of the transgene or phenotype.

Weanling transgenic mice will be fed either a normal diet or a diet high in n-6 PUFA (see above). Animals will be sacrificed at different ages (neonate, wean to 1 month, adult to 6 mos and aging—12 mos, 3-5 mice per time point will be used) and various tissues will be harvested for assessment of the expression level of transgene and determination of fatty acid composition. The results will be compared with those from wild type animals fed the same diet.

Other methods, including assessment of transgene expression (Northern blot, RNase protection assays, or in situ hybridization), analysis of fatty acid composition, measurement of eicosanoids, and statistical analyses will be carried out as described above.

In general accordance with the teaching provided above, we have made transgenic mice expressing the C. elegans fat-1 gene encoding an n-3 fatty acid desaturase. These mice are capable of producing n-3 from n-6 fatty acids, leading to enrichment of n-3 fatty acids with reduced levels of n-6 fatty acids in almost all organs and tissues, including muscles and milk, with no need of dietary n-3 fatty acid supply. This achievement supports our theory that such animals (i.e., any animal that effectively expresses or overexpresses the product of a fat-1 nucleic acid sequence) are unique research tools and a new and ideal source of n-3 fatty acids that can be used to meet the nutritional needs of humans and other animals.

To heterologously express the C. elegans n-3 fatty acid desaturase in mice, the fat-1 gene encoding this protein was modified from the original by optimization of codon usage for mammalian cells and coupled to a chicken beta-actin promoter and cytomegalovirus enhancer, which are highly active in a wide range of cell types and therefore allows high-level and broad expression of transgene in mice (Niwa et al., Gene 108:193-199, 1991; Okabe et al., FEBS Lett 407:313-319, 1997).

The expression of the fat-1 in F1 pups from transgenic founder mice and their offspring was examined by Real-Time PCR of tail DNA and by analysis of tail lipids. The transgenic mice looked normal and very healthy. Both transgenic and wild type mice were maintained at a diet high in omega-6 fatty acids (mainly linoleic acid) with very little omega-3 fatty acids (˜0.1% of total fat supplied). Feeding this n-3 depleted diet allowed us to readily identify the phenotype. Under this dietary regime, wild-type mice have little or no n-3 fatty acid in their tissues because the animals naturally cannot produce n-3 from n-6 fatty acids, whereas the fat-1 transgenic mice should have appreciable amounts of n-3 fatty acids (derived from n-6 fatty acids) in their tissues if the transgene is functional in vivo.

Since the phenotype of the transgenic mouse lines is mainly reflected by lipid profiles, we analyzed the fatty acid composition of various organs of the transgenic mice at different ages by gas chromatography-mass spectrometer.

Interestingly, the muscle of the transgenic animals has the most significant changes in these ratios, indicating the highest enzyme activity in this tissue. To date, four generations (either homozygotes or heterozygotes) of transgenic mouse lines have been examined and their tissue fatty acid profiles showed consistently high levels of n-3 fatty acids, indicating the transgene is functionally active in vivo and transmittable. Our data clearly show that the transgenic mice expressing the fat-1 gene are capable of producing n-3 fatty acids from n-6 fatty acids, resulting in enrichment of n-3 fatty acids in their organs/tissues without the need of dietary n-3 supply, which is impossible in wild type mammals.

Our findings provide a new strategy for producing n-3 PUFA-enriched foodstuff (e.g. meat, milk and eggs) by generating large transgenic animals (e.g. cow, pig, sheep, goat, rabbit, deer, chicken and other fowl (e.g., goose, duck, pheasant, and game hen)) and/or transgenic fish or other edible animals that are farmed or that reside in rivers, lakes, streams, or the sea, with the n-3 desaturase gene.

The methods used to generate the animals are described in more detail below.

Expression Vectors: The cDNA encoding the n-3 fatty acid desaturase was synthesized based on the sequence of fat-1 cloned from C. elegans (Spychalla, et al., Proc. Natl. Acad. Sci. USA 94:1142-1147, 1997) with modification of codon usage using mouse codon frequencies as reference. The synthesized fat-1 cDNA was confirmed by DNA sequencing and then cloned into a pCAGGS expression vector containing the chicken β-actin promoter and cytomegalovirus enhancer, beta-actin intron and bovine globin poly-adenylation signal. The entire insert with the promoter and coding sequence was excised with Ssp I and Sfi I and gel-purified.

Production of Transgenic Mice. Transgenic mouse lines were produced by injecting the purified Ssp I and Sfi I fragment into C57BL/6×C3H fertilized eggs. The DNA-injected eggs were transplanted to pseudo-pregnant mice (B6C3F1) to produce transgenic mice. The founder transgenic mice were identified by Real-Time PCR of tail DNA and lipid analysis of tail tissues and bred with C57BL/6J mice. We obtained seven transgenic founders. Three of them were selected to sire transgenic lines. Each has had three generations to date.

Animal feeding. Both transgenic and wild type (C57BL/6J) mice were maintained at an AIN-76A based Rodent diet containing 5% (w/w) of safflower oil. The fatty acid composition of safflower oil is as follows: 10% saturated fatty acids, 14% monounsaturated fatty acids, 76% n-6 polyunsaturated linoleic acid and 0.1% n-3 polyunsaturated alpha-linolenic acid.

Lipid Analysis. The fatty acid composition of total tissue lipids was analyzed as described previously (Kang et al., Biochem. Biophys. Acta. 1128:267-274, 1992). Lipid was extracted with chloroform/methanol (2:1, v/v) containing 0.005% butylated hydroxytoluene (as antioxidant). Fatty acid methyl esters were prepared using a 14% BF₃/methanol reagent. Fatty acid methyl esters were analyzed by gas chromatography using a fully automated HP5890 system equipped with a flame-ionization detector. The chromatography utilized an Omegawax 250 capillary column (30 m×0.25 mm I.D.). The oven program is initially maintained at 180° C. for 5 min, then ramped to 200° C. at 2° C./min and held for 48 minutes. Peaks were identified by comparison with fatty acid standards (Nu-chek-Prep, Elysian, Minn.), and area percentage for all resolved peaks was analyzed using a Perkin-Elmer M1 integrator. Fatty acid mass is determined by comparing areas of various analyzed fatty acids to that of a fixed concentration of external standard when added.

TABLE 1
Comparison of the n-6/n-3 ratios and AA/(EPA + DPA + DHA)
ratio in various organs and tissues between a wild type mouse
(WT) and a fat-1 transgenic mouse (TG)*.

| | Omega-6/Omega-3* | | AA/(EPA + DPA + DHA | |
|---|---|---|---|---|
| | WT | TG | WT | TG |
| Muscle | 49.0 | 0.7 | 11.3 | 0.4 |
| Milk*** | 32.7 | 5.7 | 15.7 | 2.5 |
| RBC | 46.6 | 2.9 | 27.0 | 1.6 |
| Heart | 22.8 | 1.8 | 14.3 | 0.9 |
| Brain | 3.9 | 0.8 | 3.6 | 0.7 |
| Liver | 26.0 | 2.5 | 12.5 | 0.9 |
| Kidney | 16.5 | 1.7 | 11.9 | 1.2 |
| Lung | 32.3 | 2.2 | 19.8 | 1.2 |
| Spleen | 23.8 | 2.4 | 17.3 | 1.5 |

*Both the wild-type and transgenic mice were 8 weeks old female and fed with the same diet.
**The n-6/n-3 fatty acid ratio is (18:2n-6 + 20:4n-6 + 22:4n-6 + 22:5n-6)/(18:3n-3 + 20:5n-3 + 22:5n-3 + 22:6n-3).
***The milk was taken from the content of stomachs of 5-day neonatal mice born from a wild type or a transgenic mother.

### Example 9

### Inhibition of Neuronal Cell Death

Construction of Recombinant Adenovirus (Ad): A recombinant Ad carrying the fat-1 gene was constructed as described previously (Kang et al., Proc. Natl. Acad. Sci. USA 98;4050-4054, 2001; see also, above). The n-3 fatty acid desaturase cDNA (fat-1 gene) used was that described above, provided in plasmid pCE8. The fat-1 cDNA was excised from the plasmid with an EcoRI/KpnI digestion, and inserted into pAdTrack-CMV vector. The construct was subsequently recombined homologously with an adenoviral backbone vector (pAdEasy 1) to generate two clones: Ad-GFP, which expresses GFP as a reporter or marker, and Ad-GFP-fat-1, which carries both the fat-1 and the GFP genes, each under the control of separate CMV promoters. Recombinant adenoviral vector DNA was digested with PacI. The linerized vector DNA was mixed with SuperFect™ (QIAGEN) and used to infect 293 cells. The recombinant viruses were prepared as high-titer stocks through propagation in 293 cells. The integrity of the constructs was confirmed by enzymatic digestion (i.e., restriction mapping) and by DNA sequencing. Purified virus was checked and its sequence confirmed again by PCR analysis.

Tissue Culture and Infection with Ad: Rat cortical neurons were prepared using standard techniques. Briefly, prenatal embryonic day 17 (E17) rat cortical neurons were dissociated and plated in poly-lysine-coated wells at 2×10⁶ cells/well. The cells were grown in Neurobasal™ Medium (NBM, Life Technologies) supplemented with 25 mM glutamic acid (Sigma Chemical Co., St. Louis, Mo.), 0.5 mM glutamine, 1% antibiotic-antimycotic solution, and 2% B27 (Life Technologies). Cultures were kept at 37° C. in air with 5% CO₂ and 98% relative humidity. The culture medium was changed every four days. After 8-10 days in culture, cells were transfected with either the Ad-GFP (control) or the Ad-GFP-fat-1 plasmids. Viral infections were carried out by adding viral particles to the culture medium. After a 48-hour incubation, cells were used for analyses of gene expression, fatty acid composition, eicosanoid production, and induction of apoptosis.

RNA Analysis: The level of fat-1 expression was determined by probing for mRNA transcripts in an RNAse protection assay using the RPA III™ kit (Ambion, Austin, Tex.). Briefly, total RNA was extracted from cultured cells using a total RNA isolation reagent (TRIzol, GIBco BRL) according to the manufacturer's protocol. The plasmid containing the fat-1 gene, pCE8, was linearized and used as a transcription template. Antisense RNA probes were transcribed in vitro using [³³P]-UTP, T7 polymerase (Riboprobe System™ T7 kit, Promega), hybridized with total RNA (15 μg) extracted from neurons, and digested with ribonuclease to remove nonhybridized RNA and probe. The protected RNA·RNA hybrids were resolved in a denaturing 5% sequence gel and subjected to autoradiography. A probe targeting the β-actin gene was used as an internal control. fat-1 mRNA was not detected in cells infected with Ad-GFP (control), but was highly abundant in cells infected with Ad-GFP-fat-1.

The cells were also examined by fluorescence microscopy. Infected cells that expressed the fat-1 gene were readily identifiable because they co-expressed GFP. Forty-eight hours after infection, 30-40% of the neurons were infected and expressed GFP. These results demonstrate that Ad-mediated gene transfer confers high expression of fat-1 gene in rat cortical neurons, which normally lack the gene.

Lipid Analysis: The fatty acid composition of total cellular lipids was analyzed as described in Kang et al. (Proc. Natl. Acad. Sci. USA 98:4050-4054, 2001). Lipid was extracted with chloroform:methanol (2:1, vol:vol) containing 0.005% butylated hydroxytoluene (BHT, as an antioxidant). Fatty acid methyl esters were prepared using a 14% (wt/vol) BF3/methanol reagent. Fatty acid methyl esters were quantified with GC/MS by using an HP-5890 Series II gas chromatograph equipped with a Supelcowax™ SP-10 capillary column (Supelco, Bellefonte, Pa.) attached to an HP-5971 mass spectrometer. The injector and detector are maintained at 260° C. and 280° C., respectively. The oven program is maintained initially at 150° C. for 2 minutes, then ramped to 200° C. at 10° C./minute and held for 4 minutes, ramped again at 5° C./minute to 240° C., held for 3 minutes, and finally ramped to 270° C. at 10° C./minute and maintained for 5 minutes. Carrier gas-flow rate is maintained at a constant 0.8 ml/min throughout. Total ion monitoring is preformed, encompassing mass ranges from 50-550 atomic mass units. Fatty acid mass is determined by comparing areas of various analyzed fatty acids to that of a fixed concentration of internal standard.

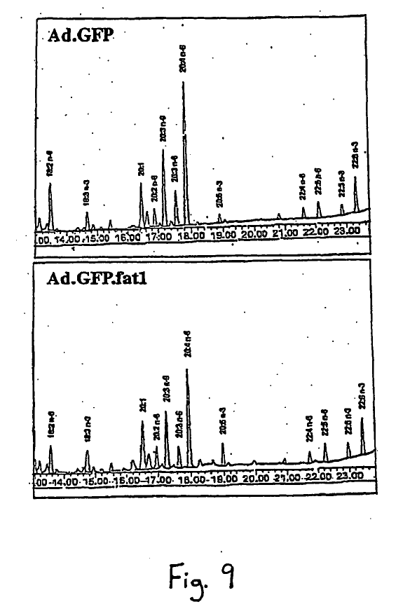

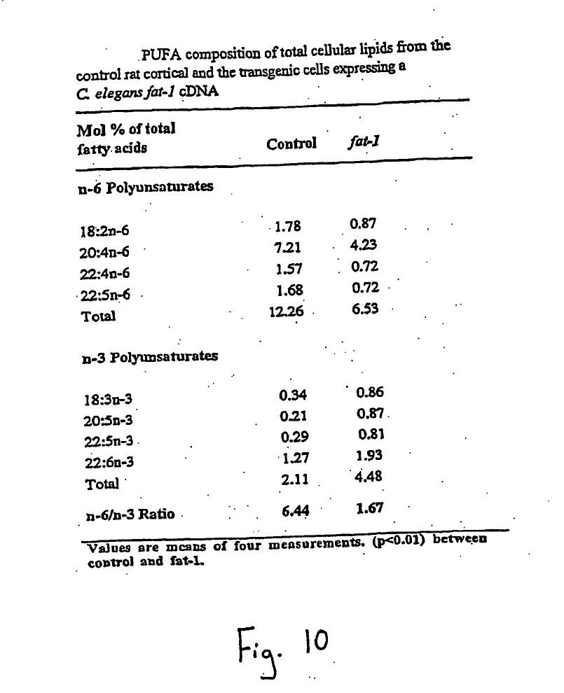

The expression of fat-1 resulted in conversion of n-6 fatty acids to n-3 fatty acids, and thus a significant change in the ratio of n-6:n-3 fatty acids. The fatty acid profile obtained from control cells is significantly different from that of cells infected with Ad-GFP-fat-1 (FIG. 9; see also FIG. 10). Cells infected with Ad-GFP show no change in fatty acid composition when compared with non-infected cells. In cells expressing the n-3 desaturase, almost all types of n-6 fatty acids were converted to the corresponding n-3 fatty acids, namely, 18:2n-6 to 18:3n-3, 20:4n-6 to 20:5n-3, 22:4n-6 to 22:5n-3, and 22:5n-6 to 22:6n-3. The change in fatty acid composition of the cells expressing the fat-1 gene resulted in reduction of the n-6:n-3 ratio from 6.4:1 in the control cells to 1.7:1 in the cells expressing the n-3 desaturase. Expression of the C. elegans n-3 fatty acid desaturase resulted in a significant increase in the levels of DHA in transfected cells. An increase in levels of EPA and ALA is observed with a concomitant decrease in AA and LA suggesting that the decrease in production of PGE₂ resulted from both the shift in the n-6:n-3 fatty acid ratio and from DHA-mediated inhibition of AA hydrolysis.

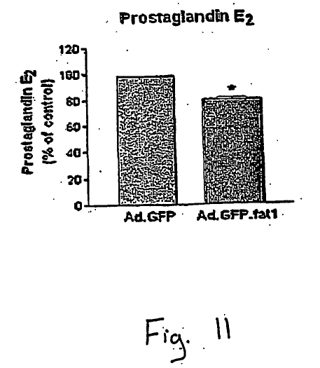

Measurement of Eicosanoids: 2-series eicosanoids may be associated with neuronal apoptosis in age-associated neurodegenerative diseases and acute excitotoxic insults such as ischemia (Sanzgiri et al., J. Neurobiol. 41:221-229, 1999; Drachman and Rothstein, Ann. Neurol. 48:792-795, 2000; Bezzi et al., Nature 391:281-285, 1998). Arachidonic acid (AA, 20:4n-6) and eicosapentaenoic acid (EPA, 20:5n-3) are the precursors of 2- and 3-series of eicosanoids, respectively. To determine whether the gene transfer-mediated alteration in the contents of AA and EPA may lead to a difference in the production of eicosanoids in the cells, we measured the production of prostaglandin E₂, one of the major eicosanoids derived from AA, in infected cells after stimulation with calcium ionophore A23187. More specifically, prostaglandin E2 was measured by using enzyme immunoassay kits (Cayman Chemical, Ann Arbor, Mich.) following the manufacturer's protocol. (The crossreactivity with prostaglandin E3 is 16%.) Cultured cells were washed with LH buffer (with 1% BSA) and incubated with the same buffer containing the calcium ionophore A23187 (5 μM). After a 10-minute incubation, the conditioned buffer was recovered and subjected to eicosanoid measurement. The amount of prostaglandin E₂ produced by fat-1 expressing cells was 20% lower than that produced by control cells (FIG. 11).

Induction of apoptosis and determination of cell growth and viability: Apoptosis was induced by growth factor withdrawal. Forty-eight hours after neurons were transfected, the culture media was changed to Neurobasal™ Medium supplemented with 25 mM glutamic acid (Sigma Chemical Co., St. Louis, Mo.) and 0.5 mM glutamine. Cytotoxicity was measured 24 hours after growth factors were withdrawn using the Vybrant™ Apoptosis Assay (Molecular Probes, Eugene, Oreg.). Briefly, cells were washed with ice-cold phosphate buffered saline (PBS) and subsequently incubated on ice for 20-30 min in ice-cold PBS containing Hoechst 33342 solution (1 ml/ml) and PI solution (1 ml/ml). A photograph was taken at the end of the incubation period.

Cell growth and viability: Cell growth and viability were determined using the MTT cell proliferation kit (Roche Diagnostic Corporation). MTT labeling reagent (100 μl) was added to each well. After 4 hours of incubation, 1.0 ml of the solubilization solution was added into each well. The cells were then incubated overnight at 37° C. and the spectrophotometrical absorbency of the solution at 600 nm was measured.

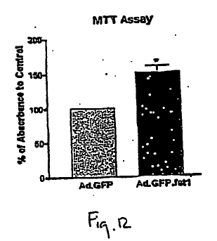

Expression of the fat-1 gene provided strong protection against apoptosis in rat cortical neurons. Hoest 33625 and PI staining of cortical cultures 24 hours after the induction of apoptosis, show that cultures infected with Ad-GFP-fat-1 underwent less apoptosis than those infected with Ad-GFP. MTT analysis indicated that the viability of Ad-GFP-fat-1 cells was significantly (p<0.05) higher than that of cells infected with Ad-GFP (FIG. 12). These results indicate that the expression of fat-1 can inhibit neuronal apoptosis and promote cell viability. The ability of the C. elegans n-3 fatty acid desaturase to inhibit apoptosis of neuronal cells highlights the importance of the n-6:n-3 fatty acid ratio in neuroprotection. Accordingly, techniques that deliver a fat-1 sequence, or a biologically active variant thereof, to neurons provide the means to quickly and dramatically balance cellular n-6:n-3 fatty acid ratio, alter eicosanoid profile (and thereby exert an anti-apoptotic effect on neuronal cells) without the need for supplementation with exogenous n-3 PUFAs. Compared to dietary intervention, this approach is more effective in balancing the n-6:n-3 ratio because it simultaneously elevates the tissue concentration of n-3 PUFAs and reduces the level of endogenous n-6 PUFAs. This method is a novel and effective approach to modifying fatty acid composition in neuronal cells, and it can be applied as a stand-alone gene therapy or as an adjuvant therapy or chemopreventive procedure (in, for example, apoplexy patients).

Data analysis, statistical analysis: Cell viability data (MTT), as well as fatty acid composition and eicosanoids levels were compared using the Student t-test. The analysis included 6 wells/group (except lipid analyses; 4 wells/group) and each experiment was repeated 3 times. The level of significance was set at p<0.05.

### Example 10

### Fat-1 Expression in Human Endothelium and Inhibition of Inflammation

To determine whether the conversion of n-6 to n-3 PUFA can be genetically conferred to primary human vascular endothelium and to study its potential protective effects against endothelial activation after cytokine stimulation, a first generation (type 5) recombinant adenoviral vector (Ad) was constructed which contained the fat-1 transgene in series with a GFP expression cassette under the control of the CMV promoter (Ad.fat-1). A GFP/β-gal adenovirus served as the control vector (Ad.GFP/β-gal). Monolayers of primary human umbilical vein endothelial cells (HUVECs) were infected withAd.fat-1 or the control Ad for 36 hours, exposed for 24 hours to 10 mM arachidonic acid, and subjected to lipid analysis by gas chromatography, surface adhesion molecule analysis by immunoassay, and videomicroscopy to study endothelial interactions with the monocytic cell line, THP-1, under laminar flow conditions.

Expression of fat-1 dramatically altered the lipid composition of human endothelial cells and changed the overall ratio of n-6 to n-3 PUFA from 8.5 to 1.4. Furthermore, after cytokine exposure (TNF-α, 5 μ/ml applied for 4 hours) fat-1 expression significantly reduced the surface expression of the adhesion molecules and markers of inflammation (E-Selectin, ICAM-1, and VCAM-1 by 42%, 43%, and 57%, respectively (p<0.001)).

We then asked whether changes in the adhesion molecule profile were sufficient to alter endothelial interactions with monocytes, the most prevalent white blood cell type found in atherosclerotic lesions. Under laminar flow and a defined shear stress of ˜2 dynes/cm², fat-1-infected HUVEC, compared to control vector-infected HUVEC, supported ˜50% less firm adhesion with almost no effect on the rolling interactions of THP-1 cells. Thus, heterologous expression of the C. elegans desaturase, fat-1, confers on human endothelial cells the ability to convert n-6 to n-3 PUFA. This effect significantly repressed cytokine induction of the endothelial inflammatory response and firm adhesion of the monocytic cell line, THP-1, under simulated physiological flow conditions. Accordingly, expression of fat-1 represents a potential therapeutic approach to treating inflammatory vascular diseases, such as atherosclerosis.

### Example 11

### n-3 Desaturase as an Anti-Arrhythmic Agent

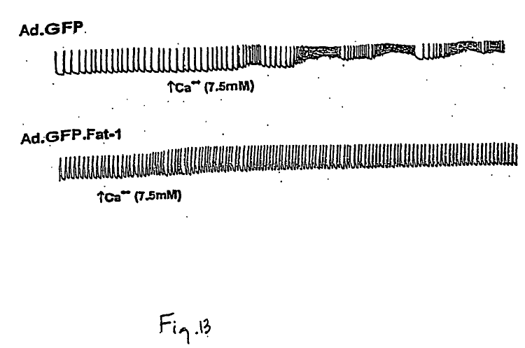

To determine whether fat-1 expression could provide an anti-arrhythmic effect, myocytes expressing the n-3 desaturase were examined for their susceptibility to arrhythmias induced by arrhythmogenic agents. Neonatal rat cardiac myocytes, grown on glass coverslips and able to spontaneously beat, were infected with Ad.GFP.fat-1 or Ad.GFP. Two days after infection, cells were transferred to a perfusion system and perfused with serum free medium containing high concentrations (5-10 mM) of calcium. These media are arrhythmogenic. During the perfusion process, myocyte contraction was monitored using a phase contrast microscope and video-monitor edge-detector. Following the high [Ca²⁺] (7.5 mM) challenge, the control cells infected with Ad.GFP promptly exhibited an increased beating rate followed by spasmodic contractions or fibrillation whereas the cells infected with Ad.GFP.fat-1 could sustain regular beating. Thus, myocytes expressing the n-3 desaturase show little, if any, susceptibility to arrhythmogenic stimuli (FIG. 13).

### Example 12

### Fat-1 Expression and Inhibition of Tumor Growth

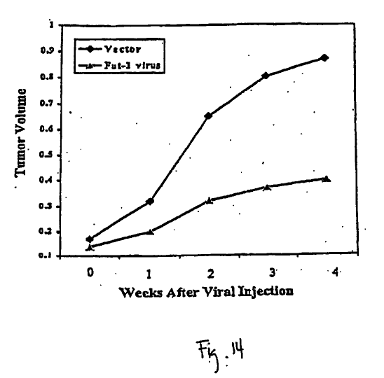

To test the effect of the gene transfer on tumor growth in vivo, we have carried out a pilot experiment in two nude mice bearing human breast cancer xenografts (MDA-MB-231). One mouse was injected intratumorally with 50 ml of Ad.GFP.fat-1 (1012 particles/ml) twice every other day. The other was injected with the control vector (Ad.GFP). The growth rate of the tumors was monitored for four weeks. The growth rate of the tumor treated with Ad.GFP.fat-1 appeared to be slower than that of the control tumor (FIG. 14).

### Example 13

### The Effect of Fat-1 Expression on Fatty Acid Composition and Growth of Human Breast Cancer Cells in Culture

Construction of Recombinant Adenovirus (Ad): A recombinant Ad carrying the fat-1 cDNA was constructed as described previously (Kang et al., Proc. Natl. Acad. Sci. USA 98:4050-4054, 2001). Briefly, the fat-1 cDNA in pCE8 (as described above) was excised from the plasmid with an EcoRI/KpnI double digest, inserted into a shutter vector and then subjected to homologous recombination with an adenoviral backbone according to the methods of He et al. (Proc. Natl. Acad. Sci. USA 95:2509-2514, 1998). Two first-generation type 5 recombinant adenoviruses were generated: Ad.GFP, which carries GFP as a reporter gene under control of the CMV promoter, and Ad.GFP.fat-1, which carries both the fat-1 and GFP genes, each under the control of separate CMV promoters. The recombinant viruses were prepared as high titer stocks through propagation in 293 cells, as described previously (Kang et al., Proc. Natl. Acad. Sci. USA 98:4050-4054, 2001). The integrity of the constructs was confirmed by enzymatic digestion and by DNA sequence analysis.

Cell Cultures and Infection with Ad.: MCF-7 cells were routinely maintained in 1:1 (v/v) mixture of DMEM and Ham's F12 medium (JRH, Bioscience) supplemented with 5% fetal bovine serum (FBS) plus antibiotic solution (penicillin, 50 U/ml; streptomycin, 50 μg/ml) at 37° C. in a tissue culture incubator with 5% CO₂ and 98% relative humidity. Cells were infected with Ad for experiments when they were grown to about 70% confluence by adding virus particles to medium without serum (3-5×10⁸ particles/ml). Initially, optimal viral concentration was determined by using Ad.GFP to achieve an optimal balance of high gene expression and low viral titer to minimize cytotoxicity. After a 24-hour incubation, the infection medium was replaced with normal culture medium supplemented with 10 μM 18:2n6 and 20:4n6. Forty-eight hours after infection, cells were used for analyses of gene expression, fatty acid composition, eicosanoid production, and cell proliferation and apoptosis.

RNA Analysis: The fat-1 transcripts were examined by ribonuclease protection assay using a RPA III™ kit (Ambion, Austin, Tex.). Briefly, total RNA was extracted from cultured cells using an RNA isolation kit (Qiagen) according to the manufacturer's protocol. The plasmid containing fat-1, pCE8, was linearized and used as transcription template. Antisense RNA probes were transcribed in vitro using ³³P-UTP and T7 polymerase (Riboprobe™ System T7 kit, Promega), hybridized with the total RNA extracted from the cancer cells, and digested with RNase to remove non-hybridized RNA and probe. The protected RNA:RNA was resolved in denaturing sequence gel and subjected to autoradiography. A probe targeting the GAPDH gene was used as an internal control.

The cells that were infected and expressed the transgene could be readily identified by fluorescence microscopy since they co-expressed the GFP (which exhibits bright fluorescence). Three days after infection, it was observed that about 60-70 percent of the cells were infected and expressed the transgene. Analysis of mRNA using a ribonuclease protection assay showed that fat-1 mRNA was highly abundant in cells infected with Ad.GFP.fat-1, but was not detected in cells infected with Ad.GFP (control). This result indicates that the Ad-mediated gene transfer could confer a high expression of fat-1 gene in MCF-7 cells, which normally lack the gene.

Lipid Analysis: To examine the efficacy of the gene transfer in modifying the fatty acid composition of the human MCF-7 cells, total cellular lipids were extracted and analyzed by gas chromatograph after infection with the Ads and incubation with n-6 fatty acids for 2-3 days. The fatty acid composition of total cellular lipids was analyzed as described (Kang et al., supra). Lipid was extracted with chloroform/methanol (2:1, vol/vol) containing 0.005% butylated hydroxytoluene (BHT, as antioxidant). Fatty acid methyl esters were prepared by using a 14% (wt/vol) BF3/methanol reagent. Fatty acid methyl esters were quantified with GC/MS by using an HP-5890 Series II gas chromatograph equipped with a Supelcowax SP-10 capillary column (Supelco, Bellefonte, Pa.) attached to an HP-5971 mass spectrometer. The injector and detector are maintained at 260° C. and 280° C., respectively. The oven program is maintained initially at 150° C. for 2 min, then ramped to 200° C. at 10° C./min and held for 4 min, ramped again at 5° C./min to 240° C., held for 3 min, and finally ramped to 270° C. at 10° C./min and maintained for 5 min. Carrier gas-flow rate is maintained at a constant 0.8 ml/min throughout. Total ion monitoring is performed, encompassing mass ranges from 50-550 atomic mass units. Fatty acid mass is determined by comparing areas of various analyzed fatty acids to that of a fixed concentration of internal standard.

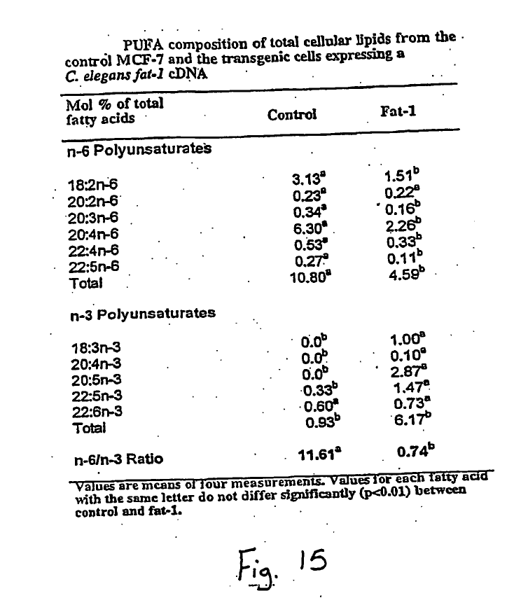

The expression of fat-1 cDNA in MCF-7 cells resulted in conversions of n-6 fatty acids to n-3 fatty acids, and a significant change in the ratio of n-6/n-3 fatty acids. The fatty acid profiles are remarkably different between the control cells infected just with the Ad.GFP and the cells infected with the Ad.GFP.fat-1 (FIG. 15). Cells infected with Ad.GFP had no change in their fatty acid profiles when compared with noninfected cells. In the cells expressing the fat-1 cDNA (n-3 fatty acid desaturase), various n-6 fatty acids were converted largely to the corresponding n3 fatty acids, for example, 18:2n6 to 18:3n3, 20:4n6 to 20:5n3, and 22:4n6 to 22:5n3. As a result, the fatty acid composition of the cells expressing fat-1 gene was changed significantly when compared with that of the control cells infected with Ad.GFP (FIG. 15), with a large reduction of the n-6/n-3 ratio from 12 in the control cells to 0.8 in the cells expressing the n-3 fatty acid desaturase.

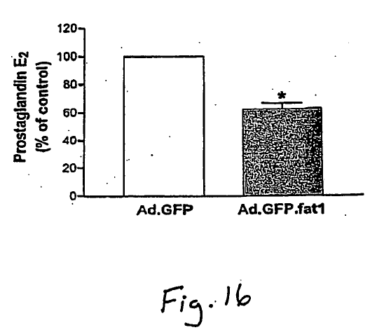

Measurement of Eicosanoids: It has been shown previously that prostaglandin E2 (PGE2), one of the major ecosanoids derived from 20:4n6 (arachidonic acid), is associated with cancer development (Rose and Connolly, Pharmacol. ther. 83:217-244, 1999; cave, Breast Cancer Res. Treat. 46:239-246, 1997). To determine whether the gene transfer-induced alteration in the contents of arachidonic and eicosapentaenoic acids can change the production of eicosanoids in the cells, we measured the production of PGE2 in the infected cells after stimulation with calcium ionophore A23187 by using an enzyme immunoassay kit that specifically detects prostaglandin E2 derived from AA with a 16% crossreactivity with prostaglandin E3 from EPA. More specifically, prostaglandin E₂ was measured by using enzyme immunoassay kits (Assay Designs, Inc) following the manufacturer's protocol. (The cross-reactivity with PGE₃ is 16%). Cultured cells were washed with PBS containing 1% BSA and incubated with serum-free medium containing calcium ionophore A23187 (5 μM). After a 10-minute incubation, the conditioned medium was recovered and subjected to eicosanoid measurement. The amount of prostaglandin E₂ produced by the fat-1 cells was significantly lower than that produced by the control cells (FIG. 16).

Analysis of Cell Proliferation and Apoptosis: To determine the effect of expression of the fat-1 gene on MCF-7 cell growth, cell proliferation and apoptosis following gene transfer were assessed. Routinely, cell morphology was examined by microscopy (dead cells appear to be detached, round and small) and total number of cell in each well was determined by counting the viable cells using a hemocytometer. In addition, cell proliferation was assessed using a MTT Proliferation Kit I (Roche Diagnostics Corporation). Apoptotic cells were determined by nuclear staining with Vybrant™ Apoptosis Kit #5 (Molecular Probes) following the manufacturer's protocol.

A large number of the cells expressing fat-1 gene underwent apoptosis, as indicated by morphological changes (small size with round shape or fragmentation) and nuclear staining (bright blue). Statistic analysis of apoptotic cell counts showed that 30-50% of cells infected with Ad.GFP.fat-1 were apoptotic whereas only 10% dead cells found in the control cells (infected with Ad.GFP). MTT analysis indicated that proliferative activity of cells infected with Ad.GFP.fat-1 was significantly lower than that of cells infected with Ad.GFP. Accordingly, the total number of viable cells in the cells infected with Ad.GFP.fat-1 was about 30% less than that in the control cells. These results are consistent with the proposition that fat-1 expression can serve as an anti-cancer agent.

Data analyses, statistical analyses: Data were presented as mean±SE. Student's T test was used to evaluate the difference between two values. The level of significance was set atp<0.05. Results

### Example 14

### Creation of Transgenic Fish

The gene encoding an n-3 fatty acid desaturase (either a wild-type gene, an optimized sequence, or other biologically active fragment or variant thereof) can be used to generate transgenic fish having a modified n-6 fatty acid content. The construct containing the desaturase gene and a promoter can be transferred into a fish by conventional gene transfer methods, such as those used with zebrafish (by sperm nuclear transplantation), as described by Jesuthasan et al. (Dev. Biol. 242: 88-95.3, 2002). The Jesuthasan method has been carried out as follows.

Sperm Nuclei Preparation: Testes were dissected from adult zebrafish (Danio rerio) males, which were sacrificed by immersion in iced water. Testes, which are located on either side of the swim bladder, are removed with fine forceps. Demembranated sperm nuclei are prepared essentially as described by Kroll and Amaya (Development 122:3173-3183, 1996), with some modifications (e.g., omission of protease inhibitors). Either lysolecithin (Sigma L4129) or digition (Sigam D5628) was used for demembranation. To check the concentration after washing, nuclei were labeled with Hoechst or Syto11 (molecular Probes) and counted on a hemacytometer. Aliquots of 10 microliters, at a concentration of approximately 100 nuclei/nl, were quick frozen in liquid nitrogen and stored at −80° C. An alternative procedure, where nuclei are demembranated by freeze-thawing, also could be used: nuclei would be washed twice in 9 ml nuclear isolation medium with 5% BSA, once in 1 ml and finally resuspended in 250 microliters before being aliquoted and quick frozen without cryoprotection.

Transgenesis Mixture: Plasmid DNA were linearized with suitable restriction enzymes, purified with Qiaquick columns (Qiagen), and diluted in sterile water to a concentration of 70 ng/μl. Sperm aliquots were thawed on ice and then mixed by pipetting up and down with a cut white tip. Five microliters of sperm suspension were transferred to a 1.5-ml Eppendorf tube, and 1 μl linearized DNA was added. For experiments with high amounts of DNA, the stock concentration can be increased while the volume added is kept at 1 μl. This combination was mixed by pipetting, kept at room temperature for 1, 5, or 20 min, then diluted with sperm dilute buffer (SDB): 250 mM sucrose, 75 mM KCl, 0.5 mM spermidine trihydrochloride, 0.2 mM sperm in tetrahydrochloride, pH 7.4. or MOH buffer (10 mM KPO₄, pH 7.2, 125 mM potassium gluconate, 5 mM NaCl, 0.5 mM MgCl₂, 250 mM sucrose, 0.25 mM spermidine trihydrochloride, 0.125 mM spermine tetrahydrochloride) to give a final volume to 500 μl. The diluted mixture was kept on ice until used.

Injection of the Sperm Nuclei: Female zebrafish were anesthetized with tricaine (SigmaA 5040), placed on a clean piece of parafilm in a petri dish, and gently squeezed to expel mature eggs. Eggs were kept in a mound, and the dish was immediately civered to prevent dehydration. A Pasteur pipette was used to transfer eggs (approximately 50 at a time) to the injection chamber, which consists of v-shaped troughs formed in 1.2% agarose in Hanks's, filled with Hanks's saline containing 0.5% BSA, to delay activation. The troughs were filled so that the eggs were just immersed—there was less than 1 mm distance between the top of the eggs and the bottom of the meniscus. This ensured efficient withdrawal of the injection needle from eggs, as eggs are held back by surface tension of the saline.

Injection needles were made by pulling thin-walled capillaries on a pipet puller, and breaking the tips with forceps so that the outer diameter is 10-15 μm (sperm nuclei have a diameter of approximately 5 μm). The needles were mounted on a holder, attached to a mechanical manipulator and filled from the tip using a microinjector. Filling was monitored with a dissecting microscope, as sperm nuclei are visible with darkfield illumination. For back-filling the mixture was drawn into tygon tubing attached to a yellow tip by using a 20 μl pipet. The tubing was then attached to the capillary and the sperm suspension extruded into the capillary and forced to the tip, using a 200 μl pipet.

Injections were be carried out with the injector by using a pressure of 3-5 psi and time of 100 ms. Sperm nuclei were injected into the animal pole region of the egg. Eggs were penetrated about 50-100 μm from the micropyle, which was visible under brightfield illumination, then rotated so that the tip was near the micropyle prior to injection. After a batch of eggs was injected, they were transferred with a Pasteur pipette to a 9-cm petri dish with 20 ml E3 (5 mM NaCl, 0.17 mM KCl, 0.33 mM CaCl₂, 0.33 mM MgSO₄) and then placed in a 28° C. incubator. Some fertilized eggs developed to adulthood, and when crossed to wild type fish, gave rise to offspring expressing the n-3 fatty acid desaturase.

Genotyping and phenotyping was performed by RT-PCR and gas chromatography (lipid analysis), respectively, as described for analysis of transgenic mice.

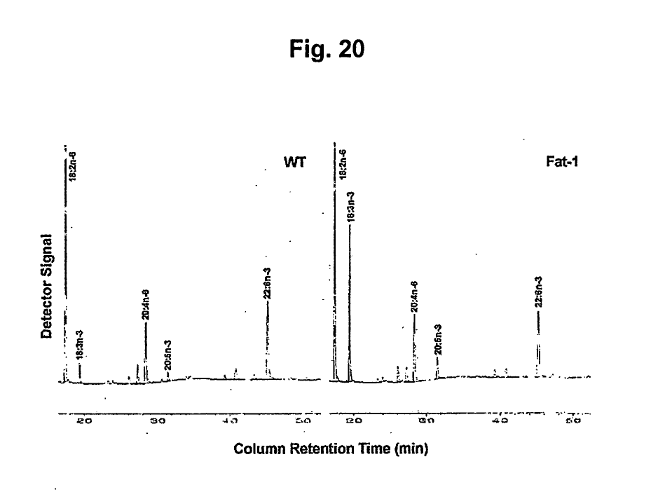

Partial gas chromatograph traces showing the differential polyunsaturated fatty acid profiles of total lipids extracted from muscle tissue of wildtype and transgenic Zebrafish expressing the fat-1 gene, modified as described in Example 8, are shown in FIG. 20. The levels of omega-3 fatty acids in the tissue of the transgenic fish were significantly higher compared to wildtype fish, and the levels of omega-6 fatty acids were markedly lower. The DNA construct used to create the transgenic fish was the same as that described in Example 8.

Other methods for introduction of foreign genes into the zebrafish germline could be used. These include injection of plasmid DNA into the early embryo, transposon-mediated gene insertion or retrovirus-mediated gene transfer (Udvadia A J and Linney E. Windows into development: historic, current, and future perspectives on transgenic zebrafish, Developmental Biology, 2003, 256: 1-17; Detrich, H. I., Westerfield, M., Zon, L. I. (Eds): The zebrafish. In Methods in Cell Biology 1999: Vol.59 & 60).

The methods described here are applicable to other teleosts, including salmon (e.g., the Atlantic salmon, Salmo salar).

For additional information, one of ordinary skill in the art can consult: Simopoulos and Cleland, World Rev. Nutr. Diet (Basel, Karger) Vol. 92, 2003; Simopoulos et al. (Eds). World. Rev. Nutr. Diet. (Basel, Karger) Vol. 83, 1998; Connor, Am. J. Clin. Nutr. 71:171S-175S, 2000, Simopoulos, Am. J. Clin. Nutr. 70:560S-569S, 1999; Salem et al., Lipids 31:S1-S326, 1996; Leaf and Weber, Am. J. Clin. Nutr. 45:1048-1053, 1987).

### Example 15

### Creation of Transgenic Pigs

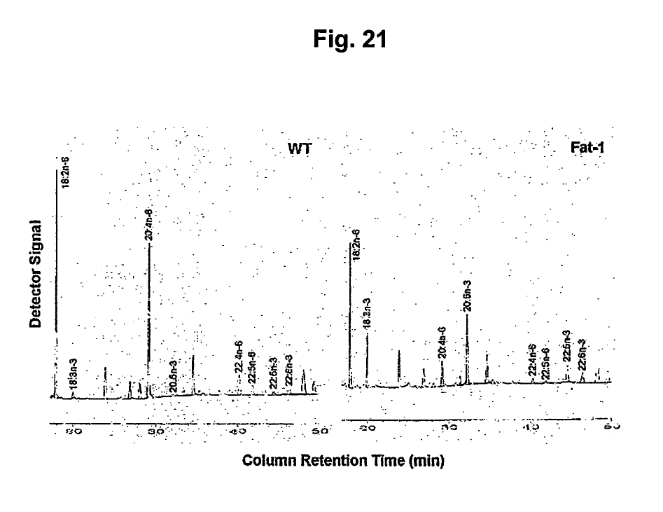

We created transgenic pigs using methods similar to those described above for the creation of transgenic mice. Transgenic pigs expressed the fat-1 gene, modified as described in Example 8. Partial gas chromatograph traces showing the differential polyunsaturated fatty acid profiles of total lipids extracted from tail tissue of wildtype and the transgenic pigs are shown in FIG. 21. The levels of omega-3 fatty acids in the tissue of the transgenic pigs were significantly higher compared to wildtype pigs, and the levels of omega-6 fatty acids were markedly lower. The DNA construct used to create the transgenic pigs was the same as that described in Example 8.

A number of embodiments of the invention have been described. Nevertheless, it will be understood that various modifications may be made without departing from the spirit and scope of the invention.

What is claimed is:

1. An isolated nucleic acid molecule comprising a sequence encoding an enzyme that desaturates an n-6 fatty acid to a corresponding n-3 fatty acid, wherein the sequence includes at least one optimized codon.

2. The isolated nucleic acid molecule of claim 1, wherein the sequence is a C. elegans fat-1 gene.

3. The isolated nucleic acid molecule of claim 2, wherein the sequence includes at least 5 and up to 150 optimized codons.

4. The isolated nucleic acid molecule of claim 2, wherein 5-10, 10-15, 15-20, 20-25, 25-30, 30-35, 35-40, 40-45, 45-50, 50-55, 55-60, 60-65, 65-70, 70-75, 75-80, 80-85, 85-90, 90-95, 95-100, 100-105, 105-110, 110-115, 115-120, 120-125, 125-130, 130-135, 135-140, 140-145, or 145-150 of the codons are optimized.

5. The isolated nucleic acid molecule of any of claim 1, comprising an optimized codon at position 6, 9, 18, 20, 22, 24, 28-30, 33-36, 47, 49, 52, 54, 58, 60, 61, 64, 67, 69-71, 73, 77, 79, 81, 86, 89, 92, 94-95, 100, 101, 105, 106, 112, 115, 118, 124, 127, 128, 131, 146, 151, 154, 161, 163, 164, 169, 178, 187, 188, 195, 197, 200, 202, 206, 210, 214, 217, 221, 223, 225, 227, 228, 232, 234, 241, 245, 255, 271, 280-282, 284, 285, 301, 303, 310, 312, 327, 362, or 370.

6. The isolated nucleic acid molecule of claim 2, comprising the sequence of the nucleic acid shown in FIG. 18.

7. The isolated nucleic acid molecule of claim 1, further comprising a nucleic acid sequence encoding a therapeutic polypeptide.

8. The isolated nucleic acid molecule of claim 1, further comprising a regulatory element or a nucleic acid sequence encoding a marker.

9. The isolated nucleic acid molecule of claim 8, wherein the regulatory element is a tissue-specific promoter.

10. An expression vector comprising the nucleic acid sequence of claim 1.

11. The expression vector of claim 10, wherein the vector is a viral vector.

12. The expression vector of claim 11, wherein the viral vector is a retroviral or adenoviral vector.

13. The expression vector of claim 10, wherein the vector is a plasmid.

14. A host cell comprising the nucleic acid molecule of claim 1.

15. A host cell comprising the expression vector of claim 10.

16. A pharmaceutical composition comprising the expression vector of claim 10 and a physiologically acceptable diluent.

17. A non-human transgenic animal comprising the nucleic acid molecule of claim 1.

18. The non-human transgenic animal of claim 17, wherein the animal is a mammal, a bird, or a fish.

19. The nonhuman transgenic animal of claim 18, wherein the mammal is a cow, a pig, or a sheep; the bird is a chicken, a turkey, a duck, a goose, or a game hen; and the fish is a salmon, trout, or tuna.

20. A food product or dietary supplement comprising the non-human transgenic animal of claim 17 or a tissue or processed part thereof.

21. A method of improving the content of n-3 fatty acids in a subject's diet, the method comprising administering to the subject the food product or dietary supplement of claim 20.

22. A method of treating a patient who has been diagnosed as having cancer, the method comprising administering to the patient a therapeutically effective amount of the nucleic acid molecule of claim 1.

23. The method of claim 22, wherein the cancer is breast cancer, colon cancer, prostate cancer, liver cancer, cervical cancer, lung cancer, brain cancer, skin cancer, stomach cancer, head and neck cancer, pancreatic cancer, a blood cancer, or ovarian cancer.

24. A method of inhibiting neuronal cell death in a subject, the method comprising administering to the subject a therapeutically effective amount of the nucleic acid molecule of claim 1.

25. The method of claim 24, wherein the subject has been diagnosed as having a neurodegenerative disease.

26. The method of claim 25, wherein the neurodegenerative disease is Alzheimer's disease, Parkinson's disease, or Huntington's disease.

27. A method of treating a subject who has, or who may develop, a condition associated with an insufficiency of n-3 polyunsaturated fatty acid (PUFA) or an imbalance in the ratio of n-3:n-6 PUFAs, the method comprising administering to the subject the nucleic acid molecule of claim 1.

28. The method of claim 27, wherein the condition is an arrhythmia, cardiovascular disease, cancer, an inflammatory disease (e.g., an inflammatory vascular disease such as atherosclerosis or a vascular condition such as restenosis), an autoimmune disease, a malformation or threatened malformation of the retina or brain, diabetes, obesity, a skin disorder, a renal disease, ulcerative colitis, Crohn's disease, or chronic obstructive pulmonary disease.

29. A method of treating a subject who has received, or who is scheduled to receive, a transplant comprising a biological organ, tissue, or cell, the method comprising administering to either the subject or the transplant, the nucleic acid molecule of claim 1.

30. A transgenic fish comprising a nucleic acid sequence encoding an enzyme that desaturates an n-6 fatty acid to a corresponding n-3 fatty acid.

31. The transgenic fish of claim 30, wherein the fish is cod or any fish of the family Gadidae, order Gadiformes; halibut; herring or any fish of the order Clupeiformes; mackerel or any fish in the family Scombridae; salmon or any fish of the Salmonidae family, including trout; perch or any fish of the family Percidae; shad or any fish of the family Clupeidae; skate or any fish of the family Rajidae; smelt or any fish of the family Osmeridae; sole or any fish of the family Soleidae; and tuna or any fish of the family Scombridae.

32. The transgenic fish of claim 30, wherein the nucleic acid sequence comprises a C. elegans fat-1 gene.

33. The transgenic fish of claim 32, wherein the C. elegans fat-1 gene contains at least one optimized codon.

34. The transgenic fish of claim 33, wherein the sequence includes at least 5 and up to 150 optimized codons.

35. The transgenic fish of claim 33, wherein 5-10, 10-15, 15-20, 20-25, 25-30, 30-35, 35-40, 40-45, 45-50, 50-55, 55-60, 60-65, 65-70, 70-75, 75-80, 80-85, 85-90, 90-95, 95-100, 100-105, 105-110, 110-115, 115-120, 120-125, 125-130, 130-135, 135-140, 140-145, or 145-150 of the codons are optimized.

36. The transgenic fish of claim 33, wherein the nucleic acid comprises an optimized codon at position 6, 9, 18, 20, 22, 24, 28-30, 33-36, 47, 49, 52, 54, 58, 60, 61, 64, 67, 69-71, 73, 77, 79, 81, 86, 89, 92, 94-95, 100, 101, 105, 106, 112, 115, 118, 124, 127, 128, 131, 146, 151, 154, 161, 163, 164, 169, 178, 187, 188, 195, 197, 200, 202, 206, 210, 214, 217, 221, 223, 225, 227, 228, 232, 234, 241, 245, 255, 271, 280-282, 284, 285, 301, 303, 310, 312, 327, 362, or 370.

37. The transgenic fish of claim 33, wherein the nucleic acid comprises the sequence of the nucleic acid shown in FIG. 18.

38. A transgenic bird comprising a nucleic acid sequence encoding an enzyme that desaturates an n-6 fatty acid to a corresponding n-3 fatty acid, and wherein the bird is bred for consumption.

39. The transgenic bird of claim 38, wherein the bird is a chicken, a turkey, a duck, a goose, or a game hen.

40. The transgenic bird of claim 38, wherein the nucleic acid sequence comprises a C. elegans fat-1 gene.

41. The transgenic bird of claim 40, wherein the C. elegans fat-1 gene contains at least one optimized codon.

42. The transgenic bird of claim 41, wherein the C. elegans fat-1 gene includes at least 5 and up to 150 optimized codons.

43. The transgenic bird of claim 42, wherein 5-10, 10-15, 15-20, 20-25, 25-30, 30-35, 35-25 40, 40-45, 45-50, 50-55, 55-60, 60-65, 65-70, 70-75, 75-80, 80-85, 85-90, 90-95, 95-100, 100-105, 105-110, 110-115, 115-120, 120-125, 125-130, 130-135, 135-140, 140-145, or 145-150 of the codons are optimized.

44. The transgenic bird of claim 41, wherein the C. elegans fat-1 gene comprises an optimized codon at position 6, 9, 18, 20, 22, 24, 28-30, 33-36, 47, 49, 52, 54, 58, 60, 61, 64, 67, 69-71, 73, 77, 79, 81, 86, 89, 92, 94-95, 100, 101, 105, 106, 112, 115, 118, 124, 127, 128, 131, 146, 151, 154, 161, 163, 164, 169, 178, 187, 188, 195, 197, 200, 202, 206, 210, 214, 217, 221, 223, 225, 227, 228, 232, 234, 241, 245, 255, 271, 280-282, 284, 285, 301, 303, 310, 312, 327, 362, or 370.

45. The transgenic bird of claim 41, wherein the C. elegans fat-1 gene comprises the sequence shown in FIG. 18.
